(19) United States
(12) Patent Application Publication
(10) Pub. No.: US 2025/0025500 A1
(43) Pub. Date: Jan. 23, 2025

(54) PHOSPHATASE INHIBITORS AS NK CELLS MODULATORS FOR THE TREATMENT OF CANCER

(71) Applicant: Kanyr Pharma Inc., Boucherville (CA)

(72) Inventors: Chu-Han FENG, Montreal (CA); Michel L. TREMBLAY, Montreal (CA)

(21) Appl. No.: 18/707,198

(22) PCT Filed: Nov. 7, 2022
(86) PCT No.: PCT/CA2022/051643

§ 371 Date: May 3, 2024

Related U.S. Application Data
(60) Provisional application No. 63/276,067, filed on Nov. 5, 2021.

(51) Int. Cl. A61K 35/17; A61K 45/06; C12N 5/0783

(52) U.S. Cl. CPC A61K 35/17; A61K 45/06; C12N 5/0646; C12N 2501/2302; C12N 2501/999

(57) ABSTRACT

The present document describes an ex vivo method of stimulating an isolated natural killer (NK) cell by treating them with an effective amount of a compound of structural Formula I, of structural Formula II, or pharmaceutically acceptable salts thereof, and stereoisomers thereof, or combinations thereof: (I) and (II). The present document also describes stimulated isolated NK cell and compositions comprising the same, and their use in methods for the prevention or treatment of cancer in subjects in need thereof.

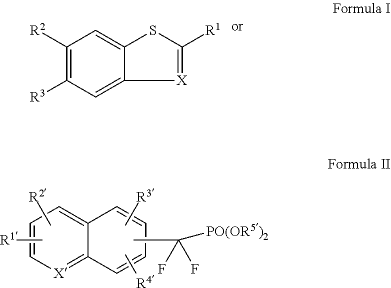

### CROSS-REFERENCE TO RELATED APPLICATIONS

This application claims priority of U.S. provisional patent application No. 63/276,067 filed on Nov. 5, 2021, the specification of which is hereby incorporated by reference in its entirety.

### BACKGROUND

### (a) Field

The subject matter disclosed generally relates to methods of ex vivo stimulation of an isolated natural killer (NK) cell comprising. More specifically, the method relates to methods of ex vivo stimulation of an isolated natural killer (NK) cell with PTPN1 and PTPN2 inhibitors and use of the isolated NK cells thus produced in the treatment of diseases, including cancer.

### (b) Related Prior Art

Natural killer cells (NK) are a type of innate lymphoid cells that functionally mirror CD8⁺ cytotoxic T cells in adaptive immunity, characterized with natural cytotoxicity against viral infected cells and tumor cells without prior antigen sensitizations. In humans, NK cells represent 5-15% of circulating lymphocytes in peripheral blood, with the majority being CD56^{dim}, cytolytic effector cells, and up to 30% in umbilical cord blood, with the majority being CD56^{bright}, cytokine producer cells. Although NK cells from these two sources display different functional advantages, both have been applied to generate chimeric antigen receptor (CAR)-engineered NK (CAR-NK) cells in treating a variety of hematological and solid tumors without causing life-threatening side effects. Novel strategies to improve CAR-NK cell persistency in vivo, infiltration into solid tumors, resistance to suppressive tumor microenvironment and to overcome technical hurdles of genetically editing NK cells are in need.

Activation of NK cell effector function is regulated at multiple stages. At “education” stage, MHC class I ligand matched inhibitory receptors potentiate NK cell response inversely through the strength of inhibitory signals to restrain NK cell response as self-tolerant to healthy cells, as well as to determine their activation threshold that is possibly related to the content, quantity and/or maturation/sorting/aggregation status of pre-formed cytolytic granules. At the target cell recognition stage, educated NK cell activation depends on synergistic signaling from a wide array of germline encoded receptors that differ within individuals. When synergized signals are received from downregulated inhibitory receptors ligands, and/or upregulated of activating receptor ligands, and/or increased co-stimulatory molecules such as cytokines, and achieve a previously set activation threshold, NK cell effector functions are initiated. This is followed by NK cell cytoskeleton rearrangement, receptor polarization, formation of cytolytic synapses and transportation and exocytosis of cytolytic effector molecules. Multiple checkpoints are placed at the effector stage to direct timely release of cytolytic granules perforin, granzymes, granulysins, death receptor ligands and cytokines to mediate “serial” killing of target cells.

Non-receptor protein tyrosine phosphatases type 1 (PTPN1 or PTP1B) and type 2 (PTPN2 or TC-PTP) are two highly homologous phosphatases that regulate immune cell development and functions. PTPN1 and PTPN2 differ from the other 16 non-receptor protein tyrosine phosphatases (NR-PTPs), as they contain two tandem phospho-tyrosine (pTyr) sites at the substrate binding domain, indicating that they can be pharmacologically inhibited simultaneously. PTPN1 and PTPN2 share around 72% amino acid sequence identity at N-terminal catalytic domains and a high degree of similarity in their tertiary protein structures, but they differ at the C-terminal domains containing ER targeted motif (PTPN1, 50 kDa and PTPN2 TC48 isoform, 48 kDa) or nuclear localization motif (PTPN2 TC45 isoform, 45 kDa). In addition, PTPN1 and PTPN2 have different substrate specificities likely due to their subcellular localizations, protein expression levels, extrinsic and context dependent signaling activators and intrinsic amino acid variants present in proximity to the catalytic domain. Besides, PTPN1 and PTPN2 display non-redundant roles in regulating the same receptor signaling and in physiological functions. Indeed, Ptpn2 null mice died at 3-5 weeks of age from severe anemia and progressive systemic inflammatory diseases whereas Ptpn1 null mice had normal life span and were resistant to diet-induced obesity and diabetes. To date, the functional roles of PTPN1 and/or PTPN2 in regulating NK cell signaling involved in activation and cytotoxic functions have not been investigated.

Hence, the inventors have found it surprising and unexpected that PTPN1 and/or PTPN2 inhibitors could also provide a stimulating effect on isolated natural killer (NK) cells, and potentiate NK cells anti-tumor cytolytic functions by increasing cytolytic granule productions and proinflammatory cytokines production after cell activation. This results in activated NK cells recognizing and killing tumor cells by augmenting perforin, granzyme A and granzyme B secretion. Therefore, these unique phenotypes and the linked signaling cascades of IL-2 in NK cells were not predictable prior to the work described herein.

### SUMMARY

According to an embodiment, there is provided an ex vivo method of stimulating an isolated natural killer (NK) cell comprising:
treating said isolated natural killer (NK) cell with an effective amount of a compound of structural Formula I, of structural Formula II, or pharmaceutically acceptable salts thereof, and stereoisomers thereof, or combinations thereof:

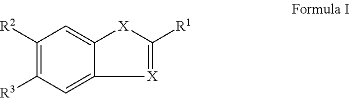

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁ 3 alkyl or C₁₋₃ haloalkyl; and
x is an integer from 0 to 2;

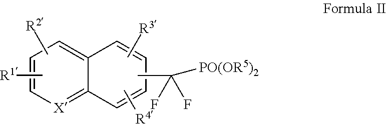

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group, each R^{5′} group is independently selected from the group consisting of H and C₁₋₃alkyl optionally substituted with 1-3 halogens, and
x is 0, 1, or 2,
   to obtain a stimulated isolated NK cell.

The compound of Formula I may be of structural Formula Ia, or a pharmaceutically acceptable salts thereof, and stereoisomers thereof:

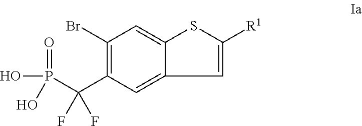

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compound of Formula I may be of structural Formula Ib, or a pharmaceutically acceptable salts thereof, and stereoisomers thereof:

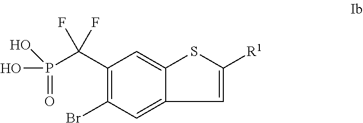

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The ex vivo method of any one of claims 1-3, wherein said compound is a compound selected from the following compounds:

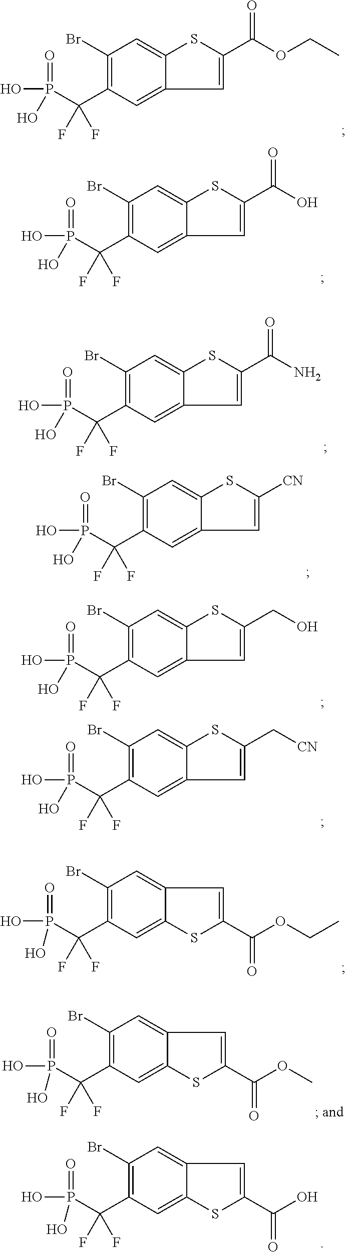

The compound of formula II may be of structural Formula IIa, or a pharmaceutically acceptable salts thereof, and stereoisomers thereof:

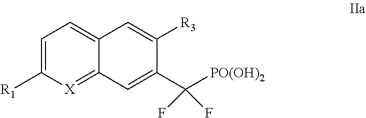

wherein
X is selected from CH and N;
R¹ is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR⁶, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂ alkyl optionally substituted with 1-3 F, (l) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂ alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R³ is halogen;
R⁶ is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, and (v) —OC₁₋₃alkyl optionally substituted with 1-3 halogens; and
x is 0, 1, or 2.

The compound may be selected from the following compounds:

| Ex. | Structure |
|---|---|
| 2 | 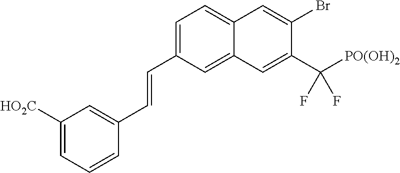 |
| 3 | 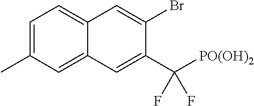 |
| 4 | 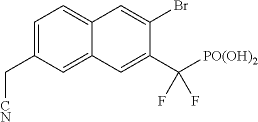 |
| 5 | 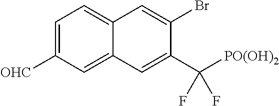 |
| 6 | 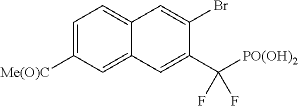 |
| 8a | 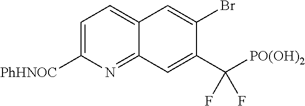 |
| 8b | 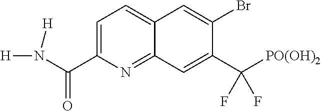 |
| 8c | 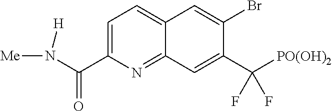 |
| 8d | 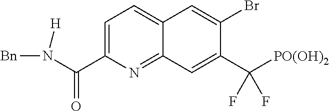 |
| 8e | 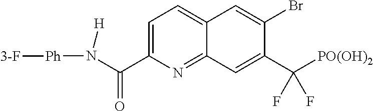 |
| 9a | 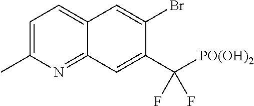 |
| 9c | 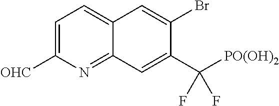 |
| 9d | 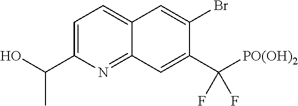 |
| 9e | 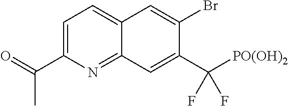 |
| 9f | 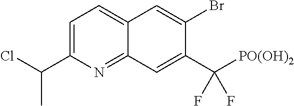 |
| 9g | 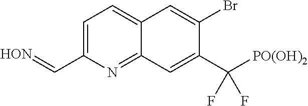 |
| 9h | 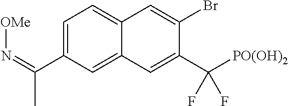 |
| L598 | 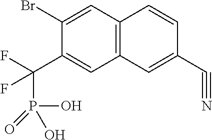 [SMILES: N#Cc1ccc2cc(Br)c(C(F)(F)P(=O)(O)O)cc2c1] |

or a pharmaceutically acceptable salt thereof.

The compound of formula (Ia) may be

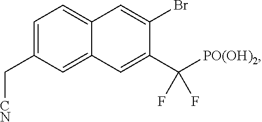

or a pharmaceutically acceptable salt thereof.

The method may further comprise a stimulation with interleukin-2 (IL-2).

According to another embodiment, there is provided a stimulated isolated NK cell prepared by the ex vivo method of the present invention.

According to another embodiment, there is provided a composition comprising the stimulated isolated NK cell of the present invention and a pharmaceutically acceptable carrier.

The stimulated isolated NK cell of the present invention, or the composition of the present invention, wherein the isolated natural killer (NK) cell may be isolated from a subject.

The stimulated isolated NK cell, or composition of the present invention, wherein the subject may be a human subject.

The stimulated isolated NK cell, or composition of the present invention, wherein the isolated natural killer (NK) cell further comprises a chimeric antigen receptor (CAR).

According to another embodiment, there is provided a method of treating cancer comprising administering to a subject in need thereof a therapeutically effective amount of the stimulated isolated NK cell of the present invention, or the composition of the present invention.

According to another embodiment, in the stimulated isolated NK cell or the composition of the present invention, the stimulated isolated NK cell or the composition is for use in preventing or treating a cancer in a subject in need thereof.

According to another embodiment, there is provided the use of the stimulated isolated NK cell or the composition of the present invention for preventing or treating a cancer in a subject in need thereof.

The cancer may be selected from the group consisting of prostate cancer, breast cancer, brain cancer, glioma, lung cancer, salivary cancer, stomach cancer, thymic epithelial cancer, thyroid cancer, ovarian cancer, multiple myeloma, leukemia, melanoma, lymphoma, gastric cancer, kidney cancer, pancreatic cancer, bladder cancer, colon cancer and liver cancer.

The method, the stimulated isolated NK cell, composition or the use of the present invention, may further comprise the administration of one or more additional compounds selected from the group consisting of:
(a) a cytotoxic agent;
(b) an antimetabolite;
(c) an alkylating agent;
(d) an anthracycline;
(e) an antibiotic;
(f) an anti-mitotic agent;
(g) an hormone therapy;
(h) a signal transduction inhibitor;
(i) a gene expression modulator;
(j) an apoptosis inducer;
(k) an angiogenesis inhibitor
(l) an immunotherapy agent.

The cytotoxic agent may be selected from the group consisting of taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, analogs or homologs thereof, and combinations thereof.

The antimetabolites may be selected from the group consisting of methotrexate, 6-mercaptopurine, 6-thioguanine, gemcitabine, cytarabine, 5-fluorouracil decarbazine, and combinations thereof.

The alkylating agent may be selected from the group consisting of mechlorethamine, thioepa chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, and combinations thereof.

The anthracycline may be selected from the group consisting of daunorubicin, doxorubicin, and combinations thereof.

The antibiotic may be selected from the group consisting of dactinomycin, bleomycin, mithramycin, anthramycin (AMC), and combinations thereof.

The anti-mitotic agent may be selected from the group consisting of vincristine, vinblastine, and combinations thereof.

The signal transduction inhibitor may be selected from the group consisting of imatinib, trastuzumab, PARPi, CDKi and combinations thereof.

The gene expression modulator may be selected from the group consisting of a siRNA, a shRNA, an antisense oligonucleotide, an HDAC inhibitor, and combinations thereof.

The immunotherapy agent may be selected from the group consisting of a monoclonal antibody, a dendritic cell (DC) vaccine, an antigen therapy, and combinations thereof.

The hormone therapy may be a luteinizing hormone-releasing hormone (LHRH) antagonist.

The apoptosis inducers may be a recombinant human TNF-related apoptosis-inducing ligand (TRAIL).

The angiogenesis inhibitors may be selected from the group consisting of sorafenib, sunitinib, pazopanib, everolimus and combinations thereof.

The monoclonal antibody may be selected from the group consisting of anti-CTLA4, anti-PD1, anti-PD-L1, anti-LAG3, anti-KIR, and combinations thereof.

The stimulated isolated NK cell may an autologous isolated NK cell from the patient in need thereof.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A illustrates a PTPN1 and PTPN2 expression level comparison in human NK-92 cell lines at mRNA analyzed by qRT-PCR. For mRNA analysis, TBP is used as a reference gene.

FIG. 1B illustrates a PTPN1 and PTPN2 protein expression level analyzed by in-direct flow cytometry.

FIG. 1C illustrates a Western blot analysis for validation on PTPN1 or PTPN2 knockdown cells.

FIG. 1D illustrates a representative figure of flow cytometry assessed shPTPN1 pool (SEQ ID NOs: 21 to 24) knockdown, shPTPN2 pool (SEQ ID NOs: 25 to 28) knockdown, shFireFly (or shFF; SEQ ID NO:29) control knockdown NK-92 cells and non-transduced NK-92 (WT) cells anti-K-562 cytolytic assays in triplicates with 3 h co-incubation time.

FIG. 1E illustrates NK-92 cells cytolysis assay against NK sensitive tumor target cells K-562 and NK resistant tumor target cells Reh for 3 hours in triplicates. NK-92 cells were pre-treated with PTPN1 and PTPN2 co-inhibitor KQ791 at 30 μM for three days in regular cell culture media containing IL-2 at 100 IU/mL.

FIG. 1F illustrates NK-92 cells cytolysis assay against NK sensitive tumor target cells K-562 and NK resistant tumor target cells Reh for 3 hours in triplicates. NK-92 cells were pre-treated with PTPN1 and PTPN2 co-inhibitor L598 at 30 μM for three days in regular cell culture media containing IL-2 at 100 IU/mL.

FIG. 1G illustrates a cord blood derived NK cells cytolysis assay against K-562 cells for 3 hours at Effector:Target=1:1 ratio. Cord blood derived NK cells were treated with KQ791 at 10 μM for five days in regular cell expansion media.

FIG. 1H illustrates a cord blood derived NK cells cytolysis assay against K-562 cells for 3 hours at Effector:Target=1:1 ratio. Cord blood derived NK cells were treated with L598 at 10 μM for five days in regular cell expansion media.

FIG. 2A illustrates a qRT-PCR analysis on relative mRNA expression of lytic granules Perforin (PRF1), Granzyme B (GZMB) and Granzyme A (GZMA) in NK-92 cells pre-treated with L598 (30 μM) or vehicle control for three days. Results were normalized against reference gene TBP except for GZMB that was normalized against B2M.

FIG. 2B illustrates a flow cytometry-based protein expression analysis of pre-formed cytolytic granule Perforin, Granzyme B and Granzyme A in resting NK-92 cells pre-treated with L598 (30 μM) or vehicle control for three days.

FIG. 2C illustrates the frequency of non-degranulation resting (CD107a⁻, perforin⁺), degranulating (CD107a⁺, perforin⁺ or CD107a⁺, granzyme B⁺), and degranulated (CD107a⁺, perforin⁻ or CD107a⁺, granzyme B⁻) NK-92 cells upon tumor target cell K-562 stimulations (E:T=1:1) or PMA/ionomycin stimulations (1 μg/mL each) or no stimulations (K-562 cell media) for six hours. Experiment was done once in triplicates.

FIG. 2D illustrates the frequency of non-degranulation resting (CD107a⁻, granzyme B⁺), degranulating (CD107a⁺, perforin⁺ or CD107a⁺, granzyme B⁺), and degranulated (CD107a⁺, perforin⁻ or CD107a⁺, granzyme B⁻) NK-92 cells upon tumor target cell K-562 stimulations (E:T=1:1) or PMA/ionomycin stimulations (1 μg/mL each) or no stimulations (K-562 cell media) for six hours. Experiment was done once in triplicates.

FIG. 2E illustrates a Mean fluorescent intensity (MFI) (Median) analysis of perforin and granzyme B expression in single and live NK-92 cell populations from FIGS. 2C (left) and 2D (right).

FIG. 2F illustrates the percentage of dead K-562 cells killed by corresponding NK-92 cells in FIG. 2D.

FIG. 2G illustrates the Kinetic of degranulation during a 3 h cytolytic assay in FIG. 1, as assessed by flow cytometry based intracellular lytic granule content analysis. NK-92 cells were pretreated with L598 (10 μM) or vehicle control for three days.

FIG. 3A illustrates a Western blot analysis of JAK/STAT signaling pathways in starved NK-92 cells with or without L598 pre-treatment to IL-2 stimulation at titrated low dosage for 15 mins.

FIG. 3B illustrates the IL-2 receptor subunits IL-2Rα (CD25) and IL-2Rβ (CD122) expression analysis on NK-92 cells with L598 (10 μM) or vehicle control with or without tumor target cells K-562 stimulation (at E:T=1:2) for four hours.

FIG. 3C illustrates the quantification of MFI (median) signal of IL-2Rα (CD25) and IL-2Rβ (CD122) expression from B.

FIG. 3D illustrates frequency of CD56 bright, cord blood derived NK cells expressing CD69 with L598 (30 μM) or vehicle control with or without tumor target cells K-562 at (E:T=1:1) stimulation for three hours.

FIG. 3E illustrates frequency of CD56 bright, cord blood derived NK cells expressing NKp30 with L598 (30 μM) or vehicle control with or without tumor target cells K-562 at (E:T=1:1) stimulation for three hours.

FIG. 3F illustrates the quantification of MFI (median) of CD69 (left) and NKp30 (right) surface expression in CD56^{bright}, CD69+ or CD56^{bright}, NKp30+ cells respectively.

FIG. 4A illustrates a qRT-PCR analysis on IFNG normalized to B2M in NK-92 cells pretreated with L598 (30 μM) or vehicle control.

FIG. 4B illustrates an ELISA analysis on IFN-γ secretion by NK-92 cells pretreated with L598 (30 μM) or vehicle control for three days, with and without tumor target cell K-562 (E:T=6:1) stimulation. Results were normalized against basal IFN-γ secretion by K-562 cells.

FIG. 4C illustrates a multi-response analysis on cytokine IFN-γ production simultaneously with degranulation by NK-92 cells with L598 (30 μM) or vehicle control pre-treatment for three days.

FIG. 4D illustrates a multi-response analysis on cytokine TNF-α production simultaneously with degranulation by NK-92 cells with L598 (30 μM) or vehicle control pre-treatment for three days.

FIG. 5A illustrates an NK-92 against K-562 cytolysis assay (3 hours) at L598 (30 μM) or vehicle control treatment for three days, subsequent removal of L598 or vehicle control for three days and subsequent re-treatment with L598 (30 μM) or vehicle control for two days.

FIG. 5B illustrates intracellular perforin (B) expression analysis in NK-92 cells upon IL-2 titration and L598 (30 μM) or vehicle control co-treatment for one day (left) and for three days (right).

FIG. 5C illustrates intracellular granzyme B expression analysis in NK-92 cells upon IL-2 titration and L598 (30 μM) or vehicle control co-treatment for one day (left) and for three days (right).

FIG. 5D illustrates the fold change of MFI (median) of perforin (top) and granzyme B (bottom) for three consecutive days with IL-2 titration and L598 co-treatment. Fold change is calculated by MFI (L598 group) divided by MFI (vehicle control group).

FIG. 5E illustrates an intracellular perforin (E) expression analysis in NK-92 cells upon IL-2 titration and withdrawal of L598 or vehicle control for one day (left) and for three days (right).

FIG. 5F illustrates an intracellular granzyme B (F) expression analysis in NK-92 cells upon IL-2 titration and withdrawal of L598 or vehicle control for one day (left) and for three days (right).

FIG. 5G illustrates the fold change of MFI (median) of perforin (top) and granzyme B (bottom) for three consecutive days with IL-2 titration and L598 withdrawal.

FIG. 6A illustrates NK-92 and K-562 cytolysis assay with IL-4 (10 ng/mL) treatment along with L598 (10 μM) or vehicle control in regular cell culture condition containing IL-2 for three days.

FIG. 6B illustrates NK-92 and K-562 cytolysis assay with TGF-β (10 ng/mL) treatment along with L598 (10 μM) or vehicle control in regular cell culture condition containing IL-2 for three days.

FIG. 6C illustrates JAK/STAT signaling analysis in NK-92 cells with L598 or vehicle control treatment and with TGF-β treatment from FIG. 6B.

FIG. 6D illustrates phosphorylated SMAD2 signaling, SOCS1 and SOCS3 signaling analysis in NK-92 cells with L598 or vehicle control treatment and with IL-4 or TGF-3 treatment from FIGS. 6A and 6B.

FIG. 7A illustrates an L598 Titration on NK-92 cytolytic activities against K-562 target cells at various E:T ratios. NK-92 cells were treated with increasing dosage of L598 for three days; no treatment is used as a negative control.

FIG. 7B illustrates an KQ791 Titration on NK-92 cytolytic activities against K-562 target cells at various E:T ratios. NK-92 cells were treated with increasing dosage of KQ791 for three days; no treatment is used as a negative control.

FIG. 8A illustrates JAK/STAT signaling pathway screen upon L598 and KQ791 titrations in NK-92 cells at regular cell culture conditions for three days.

FIG. 8B illustrates Western blot analysis on expressions of lytic granules perforin, granzyme B, granzyme A in NK-92 cells treated with L598 or KQ791 at increasing dosage for three days.

FIG. 9A illustrates phosphorylation of AKT, MAP kinase pathways upon low dosage of IL-2 stimulation for 15 mins. NK-92 cells were starved for 6 hours without L598 and cytokine treatment before IL-2 stimulations.

FIG. 9B illustrates phosphorylation of AKT, MAP kinase pathways upon low dosage of IL-15 stimulation for 15 mins. NK-92 cells were starved for 6 hours without L598 and cytokine treatment before IL-15 stimulations.

FIG. 10A illustrates NK-92 phenotype changes upon L598 treatment or vehicle control with (right) and without (left) tumor target cell K-562 stimulation (E:T=1:2) for 4 hours shown by CD69 expression.

FIG. 10B illustrates NK-92 phenotype changes upon PTPN1 knockdown or PTPN2 knockdown in comparison to shFF control (negative control targeting Firefly luciferase from Photinus pyralis; SEQ ID NO:29) with (right) and without (left) tumor target cell K-562 stimulation (E:T=1:2) for 5 hours shown by CD69 expression.

FIG. 10C illustrates NK-92 phenotype changes upon L598 treatment or vehicle control shown by NKG2D, NKp30, NKp46 (left, middle and right, respectively) expression.

It will be noted that throughout the appended drawings, like features are identified by like reference numerals.

### DETAILED DESCRIPTION

Compounds of Formula I and Formula II are inhibitors of PTPN1 and PTPN2 and are useful for the preparation of isolate natural killer (NK) cells. Such NK cell preparations may be useful in the treatment of cancer, viral infections, bacterial infections, fungal infections and parasitic infections.

According to another embodiment, the present invention also relates to methods for ex vivo treatment of NK cells harvested from a patient with compounds of Formula I or Formula II in a suitable medium in order to make those NK cells suitable for injection into a patient.

According to another embodiment, the present invention also relates to methods for ex vivo treatment of NK cells with compounds of the present invention at a concentration known to be useful to create a desired change in those cells.

According to another embodiment, the present invention also relates to a method for the incorporation of a compound of the present invention into protocols for the isolation and expansion of NK cells for use in adoptive cell transfer therapy; or for use in allogenic cell transfer therapy, such as allogenic NK cell transfer therapy. According to another embodiment, the present invention also relates to a method for the incorporation of a compound of the present invention into protocols for the isolation and expansion of NK cells for use in adoptive cell transfer therapy; or for use in autologous cell transfer therapy, such as autologous NK cell transfer therapy.

According to another embodiment, the present invention also relates to a method for the incorporation of a compound of the present invention into protocols for the generation and expansion of chimeric antigen receptor (CAR)-expressing autologous NK cells for use in adoptive cell transfer therapy; or for use in allogenic cell transfer therapy, such as allogenic NK cell transfer therapy.

According to another embodiment, the present invention also relates to methods for the treatment or control of cancer, and infectious diseases such as viral infections, bacterial infections, fungal infections and parasitic infections and related medical conditions by injecting activated NK cells or CAR-NK cells into a patient.

According to another embodiment, the present invention relates to the administration of activated NK cells or CAR-NK cells to a patient in need of such therapy by injecting such cells into the bloodstream, into a lymph node, directly into a tumor, or directly into another tissue that has been impacted by the disease the patient is being treated for.

Types of cancer that may be treated by compounds of the present invention include, but are not limited to, prostate cancer, breast cancer, brain cancer, glioma, lung cancer, salivary cancer, stomach cancer, thymic epithelial cancer, thyroid cancer, ovarian cancer, multiple myeloma, leukemia, melanoma, lymphoma, gastric cancer, kidney cancer, pancreatic cancer, bladder cancer, colon cancer and liver cancer.

Types of viral infections that may be treated by the present invention include, but are not limited to, infections caused by cytomegalovirus Epstein-Barr virus, hepatitis B, hepatitis C virus, herpes virus, human immunodeficiency virus, human T lymphotropic virus, lymphocytic choriomeningitis virus, respiratory syncytial virus, and/or rhinovirus.

Types of bacterial infections that may be treated by the present invention include, but are not limited to, infections caused by Corynebacterium, Enterococcus, Escherichia, Haemophilius, Helicobacter, Legionella, Leptospira, Listeria, Mycobacterium, Neisseria, Porphyromonas, Pseudomonus, Salmonella, Staphylococcus and Chlamydia.

Types of parasitic infections that may be treated by the present invention include, but are not limited to, infections caused by Schistosoma, Leishmania, Plasmodium, Giardia, Trypanosoma and Taenia.

Types of fungi infections that may be treated by the present invention include, but are not limited to, infections caused by Aspergillus, Blastomyces, Candida, Ringworm, and Murcormyces.

According to yet another embodiment, the invention also includes in vitro treatment of primary cells with a compound of Formula I, Formula Ia, Formula Ib, Formula II, Formula IIa, or a pharmaceutically acceptable salt thereof, in order to produce activated cells suitable for therapeutic treatment of a patient in need of immunotherapy.

### ABBREVIATIONS

Abbreviations and terms that are commonly used in the fields of organic chemistry, medicinal chemistry, pharmacology, and medicine and are well known to practitioners in these fields are used herein. Representative abbreviations and definitions are provided below:

Ac is acetyl [CH₃C(O)—], Ac₂O is acetic anhydride; ACN is acetonitrile; APC is antigen-presenting cell; Alk is alkyl; Ar is aryl; 9-BBN is 9-borabicyclo[3.3.1]nonane; Bn is benzyl; BOC is tert Butyloxycarbonyl; br is broad; CH₂Cl₂ is dichloromethane; d is doublet; DBU is 1,8-diazabicyclo[5.4.0]undec-7-ene; DC is dendritic cell; DEAD is diethyl azodicarboxylate; DIAD is diisopropylazodicarboxylate; DIBAL is diisobutylaluminum hydride; DIPEA is N,N-diisopropylethylamine; DMF is N,N-dimethylformamide; DMSO is dimethyl sulfoxide; EDAC (or EDC) is 1-ethyl-3-[3-(dimethylamino)propyl]-carbodiimide HCl; ESI is electrospray ionization; Et₃N is triethylamine; Et is ethyl; EtOAc is ethyl acetate; EtOH is ethanol; 3-F-Ph is 3-fluorophenyl; h is hours; HATU is O-(7-azabenzotriazol-1-yl)-N,N,N′,N′-tetramethyluronium hexafluorophosphate; HOAc is acetic acid; HCl is hydrochloric acid; HOBt is 1-hydroxybenzotriazole; HPLC is high performance liquid chromatography; Hunig's base is diisopropylethylamine; LiOH is lithium hydroxide; LCMS is HPLC with mass Spectral detection; LG is leaving group; m is multiplet; M is molar; mmol is millimole; Me is methyl; MeCN is acetonitrile; MeOH is methanol; MeTHF is 2-methyltetrahydrofuran; MgSO₄ is magnesium sulfate; min is minutes; MS is mass spectroscopy; MsCl is methanesulfonyl chloride; MTBE is methyl tert-butyl ether; N is normal; NaHMDS is sodium hexamethyldisiliazide; NaOAc is sodium acetate; NaOH is sodium hydroxide; NaOtBu is sodium tert-butoxide; Na₂SO₄ is sodium sulfate; NMO is N-methylmorpholine N oxide; NMP is N Methyl pyrrolidinone; NMR is nuclear magnetic resonance spectroscopy; Pd(dba)₂ is tris(dibenzylideneacetone)dipalladium; PdCl₂(Ph₃P)₂ is dichlorobis-(triphenylphosphene) palladium; PG Denotes an unspecified protecting group; Ph is phenyl; PhMe is toluene; PPh₃ is triphenylphosphine; PMB is para-methoxybenzyl; RT is room temperature; s is singlet; t is triplet; TBAF is tetrabutyl ammonium fluoride; TBS is tert-butyldimethylsilyl; tBu is tert-butyl; Tf is triflate; TFA is trifluoroacetic acid; TFAA is trifluoroacetic anhydride; THF is tetrahydrofuran; TLC is thin layer chromatography; TMEDA is N,N,N′,N′-tetramethylethylenediamine; TMS is trimethylsilyl; TPAP is tetrapropylammonium perruthenate.

### Definitions

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C3-10, the term alkyl also includes cycloalkyl groups, and combinations of linear or branched alkyl chains combined with cycloalkyl structures. When no number of carbon atoms is specified, C1-6 is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—) ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The term «composition» as used herein is intended to encompass a product comprising the specified ingredients in the specified amounts, as well as any product which results, directly or indirectly, from combination of the specified ingredients in the specified amounts. Such term in relation to pharmaceutical composition is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The terms «T cell(s)», «T lymphocyte(s)», «T cell product(s)» as used herein are intended to encompass isolated tumor-infiltrating lymphocyte (TIL), T cell receptor (TCR) engineered cell, and/or chimeric antigen receptor (CAR) engineered cell isolated by the method of the present invention. It also include different memory T cell population such as Stem central memory T_{SCM} cells, Central memory T_{CM} cells and Effector memory T_{EM} cells, that are beneficial to mount and maintain surveillance and Immune response.

The term “NK” cells refers to a sub-population of lymphocytes that is involved in non-conventional immunity. NK cells can be identified by virtue of certain characteristics and biological properties, such as the expression of specific surface antigens including CD16, CD56 and/or CD57, the absence of the alpha/beta or gamma/delta TCR complex on the cell surface, the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art.

Within the context of this invention, “potentiated”, “active,” or “activated” NK cells designate biologically active NK cells, more particularly NK cells having the capacity of lysing target cells. For instance, an “active” NK cell is able to kill cells that express an NK activating receptor-ligand and fails to express “self” MHC/HLA antigens (KIR-incompatible cells). “Potentiated”, “active,” or “activated” cells can also be identified by any other property or activity known in the art as associated with NK activity, such as cytokine (e.g., IFN-γ and TNF-α) production of increases in free intracellular calcium levels. For the purposes of the present invention, “potentiated”, “active,” or “activated” NK cells refer particularly to NK cells in vivo that are not inhibited via stimulation of an inhibitory receptor, or in which such inhibition has been overcome, e.g., via stimulation of an activating receptor.

The term “activating condition(s)” as used herein is intended to mean culture conditions that are sufficient to activate NK cells, and typically include those cytokines and chemokines, a growth factor, or ligands be present in the milieu and induce their cognate receptors in the NK cells. For example, IL-2 alone, IL-15 alone, type I interferon alone or with IL-15, IL-12 in combination with IL-15 and IL-18, and IL-2 in combination with IL-7, IL-15, SCF, FLT3L supplemented media will promote NK-cell proliferation and activation, NK cell activation methods also include agnostic antibodies towards activating receptors NKG2D alone or in combination with 2B4, towards receptors NKp30 or NKp46 or NKp44 alone, irradiated tumor cells and combinations thereof.

The terms “PTPN1” as used herein is intended to mean the tyrosine-protein phosphatase non-receptor type 1, also known as protein-tyrosine phosphatase 1B (PTP1B), and is an enzyme that is the founding member of the protein tyrosine phosphatase (PTP) family. In humans it is encoded by the PTPN1 gene. PTP1B is a negative regulator of the insulin signaling pathway and is considered a promising potential therapeutic target, in particular for treatment of type 2 diabetes. It has also been implicated in the development of breast cancer and has been explored as a potential therapeutic target in that avenue as well.

The terms “PTPN2” as used herein is intended to mean the tyrosine-protein phosphatase non-receptor type 2, also known as T-cell protein-tyrosine phosphatase (TCPTP, TC-PTP), and n humans is encoded by the PTPN2 gene.

### Compounds of Structural Formula/and Formula II

The present invention uses compounds of structural Formula I, of structural Formula II, or pharmaceutically acceptable salts thereof, and stereoisomers thereof, or combinations. The compounds structural Formula I are the following:

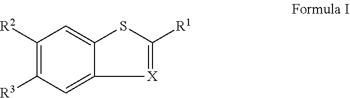

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁₋₃ alkyl or C₁₋₃ haloalkyl; and
x is an integer from 0 to 2.

The compounds of structural formula I include compounds of structural Formula Ia, or a pharmaceutically acceptable salts thereof, and stereoisomers thereof:

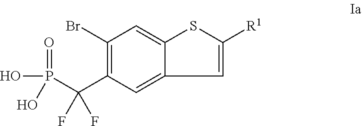

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compounds of structural formula I include compounds of structural Formula Ib, or a pharmaceutically acceptable salts thereof, and stereoisomers thereof:

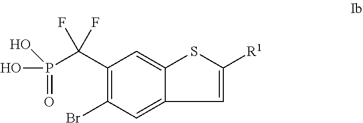

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compounds of structural Formula I may be compounds selected from the following compounds:

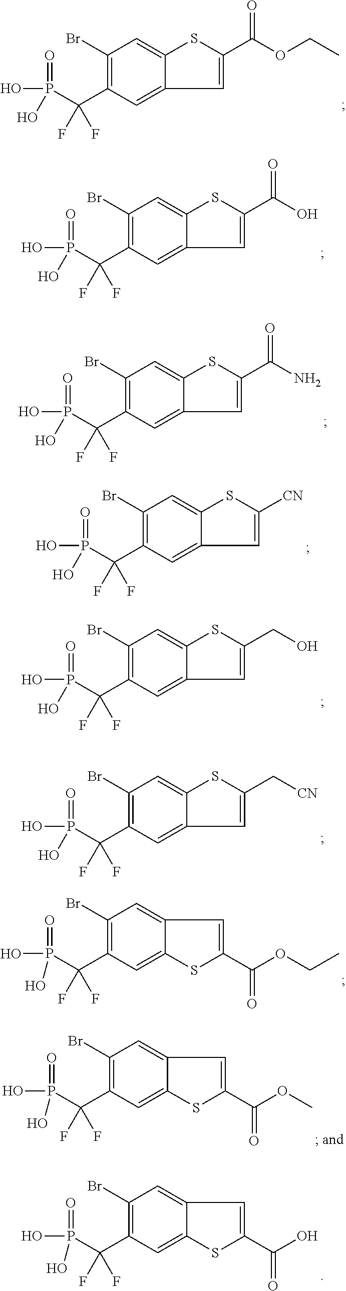

The compounds structural Formula II are the following:

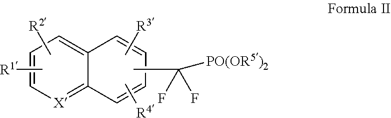

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
each R^{5′} group is independently selected from the group consisting of H and C₁₋₃alkyl optionally substituted with 1-3 halogens, and
x is 0, 1, or 2.

The compound of formula II may be of structural Formula IIa, or a pharmaceutically acceptable salts thereof, and stereoisomers thereof:

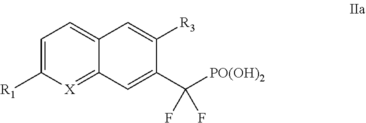

wherein
X is selected from CH and N;
R¹ is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR⁶, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂ alkyl optionally substituted with 1-3 F, (1) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂ alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R³ is halogen;
R⁶ is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, and (v) —OC₁₋₃alkyl optionally substituted with 1-3 halogens; and
x is 0, 1, or 2.

The compound of structural Formula II may be selected from the following compounds:

| Ex. | Structure |
|---|---|
| 2 | 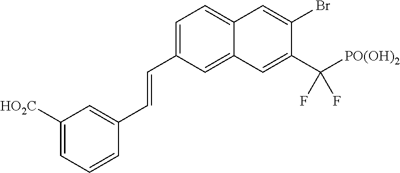 |
| 3 | 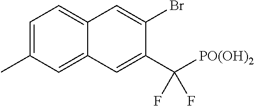 |
| 4 | 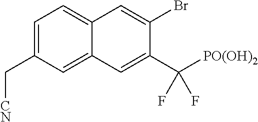 |
| 5 | 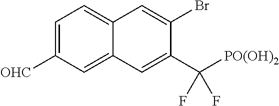 |
| 6 | 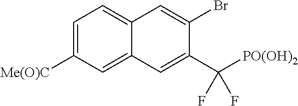 |
| 8a | 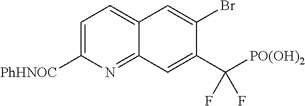 |
| 8b | 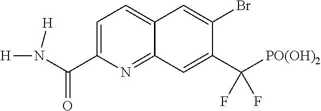 |
| 8c | 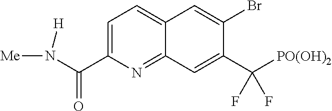 |
| 8d | 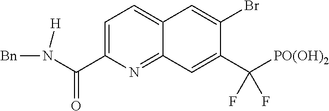 |
| 8e | 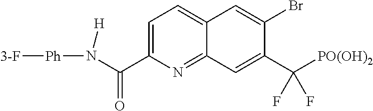 |
| 9a | 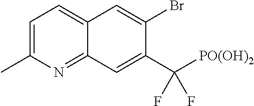 |
| 9c | 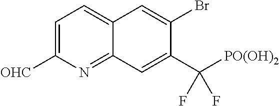 |
| 9d | 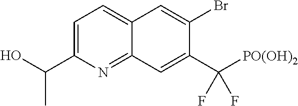 |
| 9e | 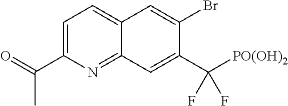 |
| 9f | 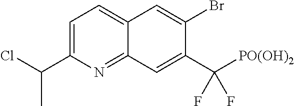 |
| 9g | 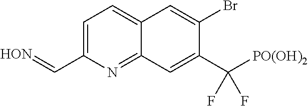 |
| 9h | 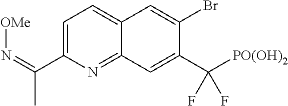 |
| L598 | 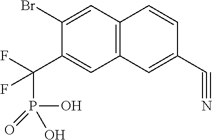 [SMILES: N#Cc1ccc2cc(Br)c(C(F)(F)P(=O)(O)O)cc2c1] |

or a pharmaceutically acceptable salt thereof.

Compounds of structural Formula I, structural Formula Ia and/or structural Formula Ib may contain one or more asymmetric centers and can thus occur as racemates and racemic mixtures, single enantiomers, diastereomeric mixtures and individual diastereomers. The present invention is meant to comprehend all such isomeric forms of the compounds of structural Formula I, structural Formula Ia and/or structural Formula Ib.

Compounds of structural Formula I, structural Formula Ia, structural Formula Ib and/or structural Formula II may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural Formula I, structural Formula Ia, structural Formula Ib and/or structural Formula II may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, Formula Ia, Formula Ib, Formula II and/or Formula IIa, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I, Formula Ia, Formula Ib and/or Formula II. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I, Formula Ia, Formula Ib, Formula II and/or Formula Ia can be prepared without undue experimentation by conventional techniques well known to those skilled in the art or by processes analogous to those described in the Schemes and Examples herein using appropriate isotopically-enriched reagents and/or intermediates.

The term “subject” as used herein, is a human patient or other animal such as another mammal with functional mast cells, basophils, neutrophils, eosinophils, monocytes, macrophages, dendritic cells, and Langerhans cells.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

### Salts and Formulations

It will be understood that, as used herein, references to the compounds of structural Formula I, Formula Ia, Formula Ib, Formula II and/or Formula Ia are meant to also include the pharmaceutically acceptable salts, and also salts that are not pharmaceutically acceptable when they are used as precursors to the free compounds or their pharmaceutically acceptable salts or in other synthetic manipulations. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural Formula I, Formula Ia, Formula Ib, Formula II and/or Formula IIa are included in the present invention as well.

The pharmaceutical compositions may be in the form of a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

### Utilities

The compounds specifically exemplified herein exhibit good efficacy in inhibiting the PTPN1 and PTPN2 enzymes, as shown by their in vitro assays. The compounds generally have an IC₅₀ value of less than 10 μM in the enzyme assay described in the Assays section, and preferably have an IC₅₀ value of less than 1 μM.

One aspect of the invention provides a method for the treatment and control of cancer, which comprises administering to a patient in need of such treatment a therapeutically effective amount of NK cells and/or CAR-NK cells that have been activated by a protocol that includes treatment with a compound of Formula I, Formula Ia, Formula Ib, Formula II and/or compounds of Formula IIa. In embodiments, the isolated NK cells may be allogenic NK cells, autologous NK cells, or combinations thereof.

A second aspect of the invention provides a method for the treatment and control of an infectious disease, which comprises administering to a patient in need of such treatment a therapeutically effective amount of NK cells and/or CAR-NK cells that have been activated by a protocol that includes treatment with a compound of Formula I, Formula Ia, Formula Ib, Formula II and/or compounds of Formula IIa. In embodiments, the isolated NK cells may be allogenic NK cells, autologous NK cells, or combinations thereof.

A third aspect of the invention provides a method for the treatment and control of immunosuppressive diseases, which comprises administering to a patient in need of such treatment a therapeutically effective amount of NK cells and/or CAR-NK cells that have been activated by a protocol that includes treatment with a compound of Formula I, Formula Ia, Formula Ib, Formula II and/or compounds of Formula IIa. In embodiments, the isolated NK cells may be allogenic NK cells, autologous NK cells, or combinations thereof.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, can be treated. However, the method can also be practiced in other species, such as avian species (e.g., chickens).

For in vitro use, the compounds of Formula I, Formula Ia, Formula Ib, Formula II or Formula IIa can be administered as a solution in water, DMSO or a mixture of water and DMSO, to a suspension of cells in a typical media such that the final concentration is about 1 nM to about 500 μM.

### Kits

Compounds of Formula I, Formula Ia, Formula Ib, Formula II and Formula IIa when being used for in vitro purposes, may be packaged for use as a crystalline solid, an amorphous solid or a lyophilized powder. Suitable quantities range from about 0.1 mg to 1 g. Ideally, the compound is packaged in a container to which a suitable solvent can be added to achieve the desired concentration of solution. Alternatively, the compound may be packaged as an aqueous solution at a fixed concentration, or as a solution in a water-soluble organic solvent at a fixed concentration. Suitable organic solvents may include DMSO, methanol, ethanol or acetonitrile, or mixtures of these solvents with water. Suitable concentrations are about 0.1 mM to about 25 mM.

The present invention includes kits encompassing the compounds of Formula I, Formula Ia, Formula Ib, formula II and/or Formula IIa, and instructions on how to use said compounds. According to an embodiment, the kit may also include appropriate cytokines, media and/or stimulatory compounds. The kit will allow a patient's cells to be conveniently activated, isolated and reinjected in a clinical setting. This treatment can be optimized to work best with current clinical therapeutic standards.

The NK cells activated with a compound of Formula I, Formula Ia, Formula Ib, Formula II and/or Formula Ia may be administered to a patient in need of immunotherapy in one or more injections. The frequency of injection and the intervals between injections will be adjusted to maximize the therapeutic response. For example, injections may occur once, twice, or more times daily, once, twice, or more times weekly, biweekly, monthly or bimonthly or at any other intervals deemed most suitable to the therapeutic benefit of the patient.

### Combination Therapy

A patient in need of immunotherapy may be treated with NK cells activated with a compound of Formula I, Formula Ia, Formula Ib, Formula II and/or Formula Ia contemporaneously with other treatments known to the medical practitioner. The use of such multiple treatments may be particularly advantageous to the patient. Such treatments may include, but are not limited to, surgical resection, radiation, chemotherapy, targeted therapy and other types of immunotherapy. Chemotherapy agents that may be used include:
a) cytotoxic agents such as taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof;
b) antimetabolites such as methotrexate, 6-mercaptopurine, 6-thioguanine, gemcitabine, cytarabine, 5-fluorouracil decarbazine;
c) alkylating agents such as mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin;
d) anthracyclines such as daunorubicin and doxorubicin;
e) antibiotics such as dactinomycin, bleomycin, mithramycin, and anthramycin (AMC);
f) anti-mitotic agents such as vincristine and vinblastine;
g) targeted therapies that may be used include, but they are not limited to: hormone therapies (such as degarelix, a luteinizing hormone-releasing hormone (LHRH) antagonist that reduces testosterone levels in prostate cancer), signal transduction inhibitors (such as imatinib, trastuzumab, PARPi, and CDKi), as well as gene expression modulators (for example the HDAC inhibitors panobinostat and belinostat), apoptosis inducers (such as recombinant human TNF-related apoptosis-inducing ligand (TRAIL)) and angiogenesis inhibitors (such as sorafenib, sunitinib, pazopanib and everolimus);
h) Immunotherapy agents that may be used include: monoclonal antibodies treatment (anti-CTLA4, anti-PD1, anti-PD-L1, anti-LAG3, anti-KIR), and dendritic cell (DC) vaccines.

Multiple treatments may also include checkpoint inhibitors or modulator of immunotherapy.

The most recognized checkpoint inhibitors are anti-PD1 and anti-CTLA4, yet in the interaction of dendritic cells with tumors cells, effector T-cells, and other immune cells, a number of protein interactions favoring or inhibiting the recognition and killing of tumor cells has been identified. For example, a dozen of those interactions have been reported to affect DC and tumors cells (K. Palucka and J. Banchereau, Nature Reviews Cancer 12:265-277). Hence the technology described herein may be conjugated to many of those additional immunotherapy technologies currently in development.

The present invention will be more readily understood by referring to the following examples which are given to illustrate the invention rather than to limit its scope.

### Example 1

### Methods

### Preparation of Compounds of the Invention

Synthetic methods for preparing the compounds of the present invention can be found in WO 2015/127548 and WO 2008/089581, incorporated herein by reference in their entireties.

### Cell Culture

Human NK cell line NK-92 was purchased from American Type Culture Collection (ATCC) and cultured in α-MEM Essential Medium (no ribonucleoside and deoxyribonucleoside) supplemented with 0.2 mM Myo-inositol, 0.02 mM Folic Acid, 0.1 mM β-mercaptoethanol, 1% of Non-essential Amino Acids, 1×L-Glutamax, 1% of Penicillin/Streptomycin, 12.5% of heat-inactivated Horse, and 12.5% of heat-inactivated Characterized Fetal Bovine Serum or FBS. The media was supplemented freshly with 200 IU/mL of IL-2 upon cell thawing for one passage and 100 IU/mL of IL-2 for cell expansion. Human tumor cell lines K-562 and Reh were purchased from ATCC and were cultured in RPM11640 Medium supplemented with 1% of Penicillin/Streptomycin and 10% of heat inactivated FBS. HEK293T/17 cells and HCT116 cells were cultured in Dulbecco's Modified Eagle's Medium (DMEM) High Glucose Medium supplemented with 1% of Penicillin/Streptomycin and 10% of FBS. All cell lines were maintained in 5% CO₂, 37° C. incubator, except that NK-92 were maintained in 6.5% CO₂ incubator at 37° C. All cell lines were maintained below 30 passages and tested regularly for mycoplasma infection using PCR.

### Human Cord Blood NK Cell Expansion and PTPN1/PTPN2 Inhibitor Treatment

Human cord blood derived NK cells (CB-NK) were kindly provided by Dr. Linda Peltier's Cellular Therapy Laboratory at McGill University Health Center (Montreal, Canada) in frozen format and were stored in liquid nitrogen tank until usage. Briefly, 1×10⁷ cells were resuspended in frozen medium consisting of ImmunoCult T Cell Medium supplemented with 10% DMSO final concentration. Frozen CB-NK cells were thawed, and ex vivo expanded at Goodman Cancer Research Center (GCRC) for five days in xeno-free ImmunoCult T cell expansion media supplemented with 10% PlasmaLyte A and freshly with a cytokine cocktail of IL-2 (5 ng/mL), IL-7 (5 ng/mL), IL-15 (5 ng/mL), SCF (15 ng/mL) and FLT3/L (10 ng/mL) every two days. After adaptation for cell expansion, CB-NK, cells were plated in complete media in presence of the PTPN1 and PTPN2 co-inhibitor L598 (7-bromo-6-phosphono(difluoro-methyl)-3-napthalenonitrile; Montalibet J, et al., J Biol Chem 2006; 281:5258-66) at 10 μM or vehicle control for five days. On day three, inhibitor L598 and fresh cytokine cocktails were supplemented to the cell media. NK cell population purity and immunophenotype profiling of major activating and inhibitory receptors were assessed upon receiving shipments, after ex vivo expansion for five days and after anti-tumor cytolytic assay.

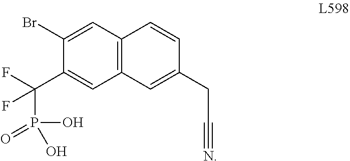 [SMILES: N#CCc1ccc2cc(Br)c(C(F)(F)P(=O)(O)O)cc2c1]

The compound KQ791 (from WO2008/089581) is also used in a number of experiments described herein.

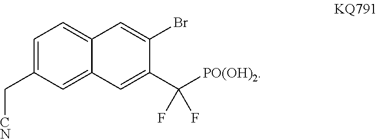

### Lentiviral Transduction and Cell Sorting

shPTPN1 or/and shPTPN2 stable knockdown NK-92 cells were generated with lentiviral spinoculation. Briefly, four constructs of shPTPN1 or shPTPN2 (SEQ ID NOs: 21 to 28) were combined at equal amount to transfect HEK293/T17 cells with Lipofectamine 2000™ (Invitrogen™) according to the manufacture's protocol; cell supernatant were collected 24 h and 48 h post-transfection and were combined and concentrated with Viro-PEG Lentivirus Concentrator™ (OZ Bioscience™) according to the manufacture's protocol. Concentrated lentivirus particles were resuspended in virus storage buffer (20 mM PIPES, 75 mM NaCl, 2.5% sucrose, pH at 6.5) and were frozen at −80° C. until usage. Lentiviral tittering was performed with HCT116 cells and assessed by % of viable GFP+ cells using flow cytometry. Spinoculation of NK-92 cells were performed in a RetroNectin® (TaKaRa™) coated 24-well plate format. NK-92 cells were stimulated with cytokines IL-2 (10001U/mL) and IL-12 (100 ng/mL) for two hours before spinoculation. Following cytokine stimulation, NK-92 cells were plated at 250,000 cells/well to RetroNectin® coated 24-well plate in 0.5 mL of serum and antibiotic free media. BX795 (working concentration at 6 μM) was added to NK-92 cells to inhibit NK cell anti-viral responses. Lentiviral particles were added at M.O.I.=30 to NK-92 cell per well. Then, the plate was centrifuged at 1000 g for 1 hour at 32° C. and incubated at 5% CO₂, 37° C. incubator for 4-6 hours, followed by a second centrifugation at 1000 g for 1 hour at 32° C. After the second centrifugation, the supernatant was removed, and fresh complete NK-92 cell media was added at 1 mL per well. Transduced NK-92 cells were cultured at 5% CO₂, 37° C. incubator for three days. Viable and GFP^{high} transduced cells were sorted by flow cytometry and expanded in regular NK-92 cell culture condition.

### Immunoblotting and Cytokine Stimulation Assay

Total cell lysate was prepared with modified RIPA buffer (1M Tris-HCl (pH 7.5), 5M NaCl, 0.25% sodium deoxycholate, 10% NP-40 and 10 mM sodium fluoride) supplemented with ethylenediaminetetraacetic acid (EDTA)-free protease inhibitor (Roche™) and 2 mM sodium orthovanadate (Sigma-Aldrich™). Protein levels were quantified by bicinchoninic acid (BCA) assay. Protein lysate was separated by 10% SDS-PAGE and Western analysis was performed using primary antibodies listed in the tables below followed with horseradish peroxidase (HRP, 1:10,000, Jackson ImmunoResearch™) conjugated secondary antibodies.

TABLE 1
RT-qPCR Human Primer Sequences

| Reference | | |
|---|---|---|
| Genes | Forward Sequence | Reverse Sequence |
| B2M | SEQ ID NO: 1: | SEQ ID NO: 2: |
| | AGTATGCCTGCCGTGTGAAC | TCATCCAATCCAAATGCGGC |
| HPRT | SEQ ID NO: 3: | SEQ ID NO: 4: |
| | AGGACCTCTCGAAGTGTTGG | GGCTTTGTATTTGGCTTTTCC |
| TBP | SEQ ID NO: 5: | SEQ ID NO: 6: |
| | TTAACTTCGCTTCCGCTGGC | TGCGCTGGAACTCGTCTCAC |
| Target | | |
| Genes | Forward Sequence | Reverse Sequence |
| PTPN1 | SEQ ID NO: 7: | SEQ ID NO: 8: |
| | GGCCCGTCATGGAGATGGAA | GCTTGGCCACTCTACATGGGA |
| PTPN2 | SEQ ID NO: 9: | SEQ ID NO: 10: |
| | TCGAGCGGGAGTTCGAAGAG | CACGACTGTGATCATATGGGCT |
| Perforin | SEQ ID NO: 11: | SEQ ID NO: 12: |
| | CGCCTACCTCAGGCTTATCTC | CCTCGACAGTCAGGCAGTC |
| Granzyme A | SEQ ID NO: 13: | SEQ ID NO: 14: |
| | TCAGGTTGATTGATGTGGGACAG | GACCATGTAGGGTCTTGAATGAGGA |
| Granzyme B | SEQ ID NO: 15: | SEQ ID NO: 16: |
| | TGGGGGACCCAGAGATTAAAA | TTTCGTCCATAGGAGACAATGC |
| Granzyme K | SEQ ID NO: 17: | SEQ ID NO: 18: |
| | ATCAACACATTTCATCTGGGCTTC | AAACGTGATGTCCGCCATACTG |
| IFN-γ | SEQ ID NO: 19: | SEQ ID NO: 20: |
| | AAGTGATGGCTGAACTGTCG | GCAGGCAGGACAACCATTAC |

TABLE 2
Flow Cytometry Antibodies

| Targets | Fluorochrome | Clone | Suppliers |
|---|---|---|---|
| CD3 | Biotin-APC/Cy7 and Pacific | HIT3a | BioLegend ® |
| | Blue | | |
| CD56 | BV605 | HCD56 | BioLegend ® |
| CD34 | BUV 737 and PE | 563 | BD ® |
| | | | Biosciences |
| CD45 | BUV 395 | HI30 | BD ® |
| | | | Biosciences |
| CD66b | Biotin-APC/Cy7 and PE/Cy7 | G10F5 | BioLegend ® |
| CD19 | Biotin-APC/Cy7 and BV711 | HIB19 | BioLegend ® |
| CD33 | Biotin-APC/Cy7 and APC | WM53 | BioLegend ® |
| CD14 | Biotin-APC/Cy7 and FITC | HCD14 | BioLegend ® |
| CD16 | FITC | 3G8 | BioLegend ® |
| CD16 | Alex 700 | 3GB | BioLegend ® |
| CD2 | PerCP/Cy5.5 | RPA-2.10 | BioLegend ® |
| CD69 | PE/Cy7 | FN50 | BioLegend ® |
| DNAM-1 | PE | 11A8 | BioLegend ® |
| 2B4 | APC | 2-69 | BioLegend ® |
| NKG2D | FITC | 1D11 | BioLegend ® |
| NKp44 | PE/Cy7 | P44-8 | BioLegend ® |
| NKp46 | PE | 9E2 | BioLegend ® |
| NKp30 | APC | P30-15 | BioLegend ® |
| CD158a | FITC | HP-MA4 | BioLegend ® |
| CD158b | FITC | DX27 | BioLegend ® |
| CD158e1 | FITC and APC | DX9 | BioLegend ® |
| CD94 | PE/Cy7 | DX22 | BioLegend ® |
| CD94 | BV421 | HP-3D9 | BD ® |
| | | | Biosciences |
| NKG2A | BV711 | 131411 | BD ® |
| | | | Biosciences |
| TIGIT | PE | A15153G | BioLegend ® |
| LAIR-1 | Alex 647 | NKTA255 | BioLegend ® |
| CD57 | APC | HNK-1 | BioLegend ® |
| NKG2C | BB700 | 134591 | BD ® Biosci |
| CD25 | Alexa 700 | BC96 | BioLegend ® |
| CD122 | APC | Tu27 | BioLegend ® |
| CD132 | PE | TUGh4 | BioLegend ® |
| CD107a | PE/Cy7 | H4A3 | BioLegend ® |
| IFN-γ | BV786 | 4S.B3 | BD ® |
| | | | Biosciences |
| TNF-α | BV421 | Mab11 | BioLegend ® |
| GrzA | APC | CB9 | BioLegend ® |
| GrzB | BV405 | GB11 | BD ® |
| | | | Biosciecnes |
| GrzB | PE | QA18A28 | BioLegend ® |
| PRF1 | FITC | B-D48 | BioLegend ® |

TABLE 3
Western Blot Antibodies

| | Catalog | Host | Dilu- | |
|---|---|---|---|---|
| Targets | Number | Species | tion | Suppliers |
| Phospho-STAT1 | 7649S | Rabbit | 1:1000 | Cell Signaling |
| (Y701) | | | | Technology ® |
| | | | | (CST) |
| STAT1 | 9172T | Rabbit | 1:1000 | CST ® |
| Phospho-STAT2 | ab53132 | Rabbit | 1:1000 | Abcam ® |
| (Y690) | | | | |
| STAT2 | ab134192 | Rabbit | 1:2000 | Abcam ® |
| Phospho-STAT3 | 9131S | Rabbit | 1:1000 | CST ® |
| (Y705) | | | | |
| STAT3 | 4904S | Rabbit | 1:2000 | CST ® |
| Phospho-STAT4 | 5267S | Rabbit | 1:1000 | CST ® |
| (Y693) | | | | |
| STAT4 | 2653 | Rabbit | 1:1000 | CST ® |
| Phospho-STAT5 | 9351S | Rabbit | 1:1000 | CST ® |
| (Y694) | | | | |
| STAT5 | 94205S | Rabbit | 1:2000 | CST ® |
| Phospho-STAT6 | 9361 | Rabbit | 1:1000 | CST ® |
| (Y641) | | | | |
| STAT6 | 5397S | Rabbit | 1:2000 | CST ® |
| Phospho-AKT | 9271S | Rabbit | 1:1000 | CST ® |
| (S473) | | | | |
| AKT | 9272 | Rabbit | 1:1000 | CST ® |
| Phospho-p38 | 9211S | Rabbit | 1:1000 | CST ® |
| (T180/Y182) | | | | |
| p38 | 9212S | Rabbit | 1:1000 | CST ® |
| Phospho-p44/42 | 9106S | Rabbit | 1:1000 | CST ® |
| (T202/Y204) | | | | |
| p44/42 | 9102S | Rabbit | 1:1000 | CST ® |
| Phospho-SMAD2 | 3108S | Rabbit | 1:1000 | CST ® |
| (S465/467) | | | | |
| SMAD2 | 3103S | Mouse | 1:1000 | CST ® |
| SOCS1 | 3950S | Rabbit | 1:1000 | CST ® |
| SOCS3 | 2923S | Rabbit | 1:1000 | CST ® |
| PTPN1 | 610140 | Mouse | 1:1000 | BD ® |
| | | | | Biosciences |
| PTPN2 | Homemade, | Mouse | 1:2000 | Homemade |
| | Clone 3E2 | | | |
| Granzyme A | ab209205 | Rabbit | 1:1000 | Abcam ® |
| Granzyme B | 4275 | Rabbit | 1:1000 | CST ® |
| Perforin | ab47225 | Mouse | 1:1000 | Abcam ® |
| Vinculin | 66305-1-IG | Mouse | 1:10000 | Thermo |
| | | | | Fisher ® |
| Actin | A2066 | Rabbit | 1:1000 | Sigma- |
| | | | | Aldrich ® |

For cytokine stimulation assay, NK-92 cells were pre-treated with L598 (30 μM) or vehicle control for three days. Before IL-2 stimulation, NK-92 cells were washed once with serum depleted cell media (no IL-2) and were starved for six hours in presence of L598 or vehicle control. After starvation, NK-92 cells were stimulated with IL-2 across a gradient concentration ranging from 0 to 90 IU/mL for 15 mins at 37° C. After incubation, cells were directly lysed with 2× concentrated TNE buffer and then boiled in 1× Laemmli Buffer to prepare cell lysates. Two independent experiments were performed.

### Degranulation Assay

NK-92 cells pre-treated with L598 (30 μM) or vehicle control were stimulated with K-562 tumor target cells [effector: target (E:T) at 1:1] or PMA/lonomycin (1 μg/mL each) or no-stimulation control with K-562 complete media, respectively. Anti-CD107a or LAMP-1 antibody (1:100, PE/Cy7 conjugated, clone H4A3, from BioLegend®) was added to cells at the start of degranulation assay. Golgi stop (containing Monensin, 1 μM) and Golgi plug (containing Brefeldin A, 4 μg/mL) were added to cells one hour after degranulation assay started and were kept until the end of the 6-hour-degranulation assay. Intracellular staining of GrB (1:40, PE conjugated, clone QA18A28, from BioLegend®) and PRF (1:40, FITC conjugated, clone B-D48, from BioLegend®) were performed with the Fixation/Permeabilization Solution Kit (BD® Biosciences) according to the manufacture's protocol. Cells were stained with fixable viability dye (1:2000) for 20 mins on ice and in dark, and blocked with FcBlock (1:20, diluted in 1× Perm/Wash buffer, Human truStain FcX from BioLegend®) for 5 mins at room temperature and in dark before intracellular staining. Cell gating and percentage of population quantification was based on no-stimulation control. For kinetics of degranulation assay, measurements were taken over three hours at 15 mins, 30 mins, 60 mins, 120 mins and 180 mins, with L598 (30 μM, three days) treated group and vehicle control group under the condition of tumor target cell K-562 stimulation (E: T at 1:1) or no stimulation. Intracellular cytolytic granule contents GrA, GrB and PRF were measured as mentioned previously.

### Cytolytic Assay and Supernatant Collection

NK-92 anti-tumor cytolysis function was assessed by flow cytometry-based analysis. Tumor target cells K-562 and Reh were labelled with cell tracker CFSE (2.5 M) or CMRA (5 μM) according to manufacturer's protocol at a maximum of one day before cytolysis experiment. On the experiment day, labelled tumor target cells were washed once with PBS and resuspend to desired cell concentration in complete cell media. NK-92 cells were washed and resuspended in complete media without cytokines to desired cell concentration. NK-92 cells and tumor target cells were plated at various effector: target (E:T) ratios with basal cell numbers at 50,000 cells per well in round-bottom 96-well plate and were co-cultured for 3 hours in 5% CO₂, 37° C. incubator. After co-culture, cells were washed once with ice-cold PBS and stained with fixable viability dyes: Aqua (Invitrogen™) or UV Blue (Invitrogen™) or eFluor 780 (eBioscience Invitrogen™) at 1:2000 in dark for 20 mins at 4° C. Stained cells were washed twice with ice cold PBS and were fixed with 1% Paraformaldehyde (PFA) for 1 hour or 2% PFA for 30 mins in dark at 4° C. Flow cytometry sample acquisition was performed with BDO LSRFortessa™ with a UV laser at Flow Cytometry Core Facility, McGill University, and data was analyzed with FlowJo™ 10.7.1 and summarized with Prism™ 9 (GraphPad™). Calculation for % of specific cytolysis was followed by the equation:

$$\% of Specific Cytolysis = \frac{\begin{matrix}100 \times ( \% of Dead Target Cells - \\ \% of Basal Death of Target Cells )\end{matrix}}{100 - \% of Basal Death of Target Cells}$$

### Quantitative Real-Time RT-PCR

Total RNA was isolated with Qiagen™ RNeasy® Mini Kit™ without DNase I on column digestion, according to the manufactures protocol. Purified total RNA was resuspended in Dnase free, Rnase free, Protease free sterile water and quantified with NanoDrop™. A260/280 was above 2.0 (data not shown). RNA integrity was checked by gel electrophoresis. 500 ng of RNA was reverse transcribed into cDNA using the SuperScript™ III Reverse Transcriptase Kit™ (Thermo Fisher Scientific™), according to the manufacturer's protocol with random primer and Oligo(dT) 20 primer. Synthesized cDNA was treated with RNaseH. qRT-PCR was performed with BioRad™ CFX96 Real Time PCR Detection™ system using FastStart Essential DNA Green Master™ according to the manufacturer's instructions. TBP and B2M was used as housekeeping genes. Delta delta Cq method was used for analyzing relative expression of gene of interests with normalization to housekeeping genes.

### ELISA

Concentration of secreted IFN-γ in supernatants of NK-92 co-cultured with K-562 cells (E:T at 6:1) or no-stimulation control (K-562 cell media) for four hours was analyzed with Human IFN-γ ELISA Kit (R&D, DIF50) according to the manufacture's protocol. Results were normalized against IFN-γ secreted by K-562 alone treated with NK-92 cell media (no cytokine) for four hours.

### Example 2

### PTPN1 and PTPN2 are Potential Targets to Enhance NK Cell Anti-Tumor Cytolysis Functions

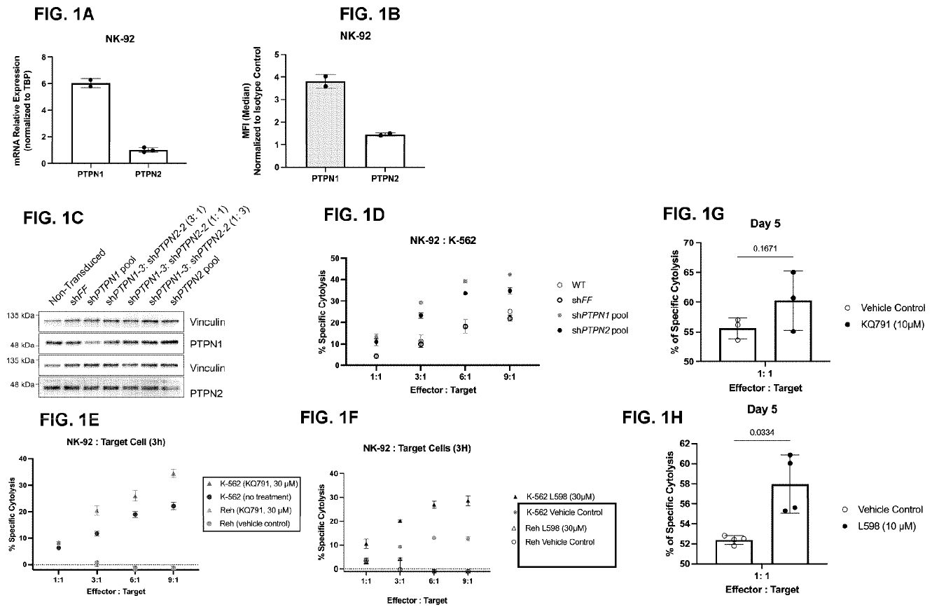

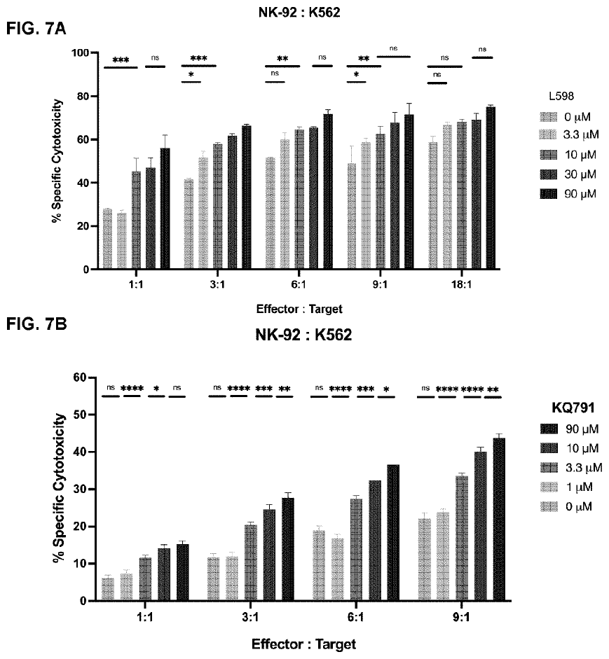

PTPN1 and PTPN2 are both ubiquitously expressed non-receptor phosphatases, while PTPN2 is found more abundant in hematopoietic cells. In NK-92 cells, PTPN1 is expressed at higher mRNA and protein levels than PTPN2 (FIG. 1B). Lentiviral transduced stable knockdown shPTPN1 pool (SEQ ID NOs: 21 to 24) and shPTPN2 pool (SEQ ID NOs: 25 to 28) NK-92 cells were generated separately (FIG. 1C) and it was found that deficiency of either of the two phosphatases improved NK-92 direct cytolysis against NK-sensitive chronic myelogenous leukemia cell line K-562 in vitro, relating them as negative regulators of NK cell cytolytic functions (FIG. 1D). Notably, PTPN1 deficiency is more potent than PTPN2 deficiency in enhancing NK-92 cell cytolysis, indicating that their quantitative expressions as well as sub-cellular localization may provide specific substrates access to the two proteins, and be important factors in regulating NK cell functions. To study the cooperative effects of PTPN1 and PTPN2 in NK-92 anti-tumor cytolysis, the PTPN1 and PTPN2 pharmacological inhibitor L598 that binds specifically to their catalytic domain but no other protein tyrosine phosphatases [L598 (7-bromo-6-phosphono(difluoro-methyl)-3-napthalenonitrile; Montalibet J, et al., J Biol Chem 2006; 281:5258-66)] was used. It was observed that there was a gradual increasing of tumor cytolysis by NK-92 cells against K-562 as the dosage of L598 or KQ791 is increased up to 90 μM (FIGS. 7A and B). NK-92 viability was not affected by the PTPN1 and PTPN2 co-inhibitor treatment at high molar concentration while NK-92 appeared to proliferate slightly faster at various IL-2 concentration upon treatment with L598, compared to the vehicle control group (not shown). Furthermore, inhibition of PTPN1 and PTPN2 with L598 did not improve cytolysis functions towards NK resistant target cells Reh (FIGS. 1E and F), indicating that additional negative regulations are in control of PTPN1 and PTPN2 regulated NK cell cytolysis functions resulting in lower risks of “bystander” killing. To examine whether this observation with NK-92 cell line could be repeated with human primary NK cells, cord blood derived NK cells, that are CD56^{bright} like the NK-92 cells, were treated with L598 or KQ791 at 10 μM for five consecutive days. Similar enhancement of cytolysis in K-562 targeted killing was observed (FIGS. 1F-H).

### Co-Inhibition of PTPN1 and PTPN2 Increases Lytic Granules Expression and Persistent Degranulation

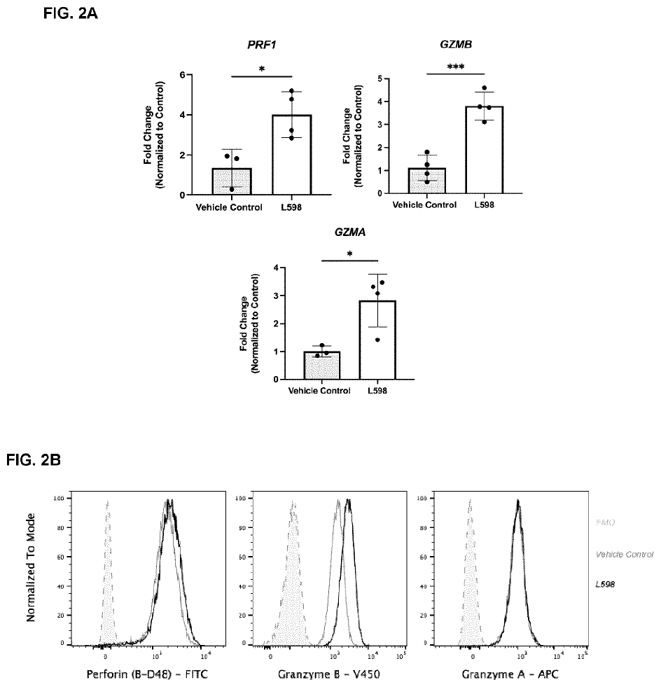

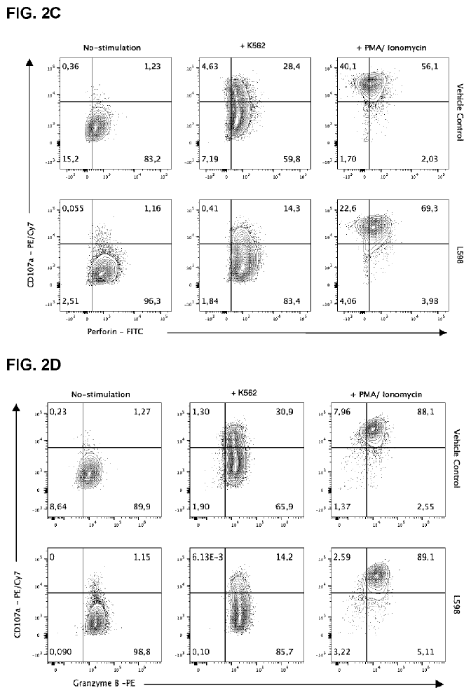

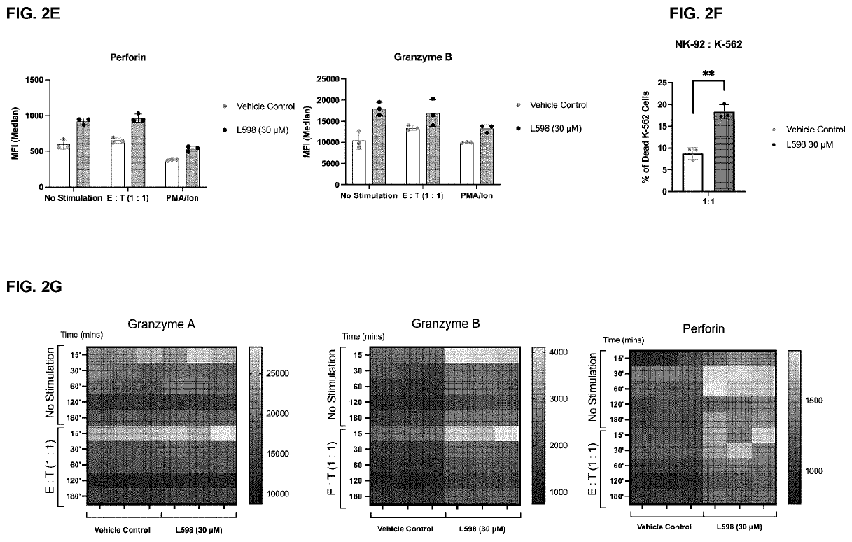

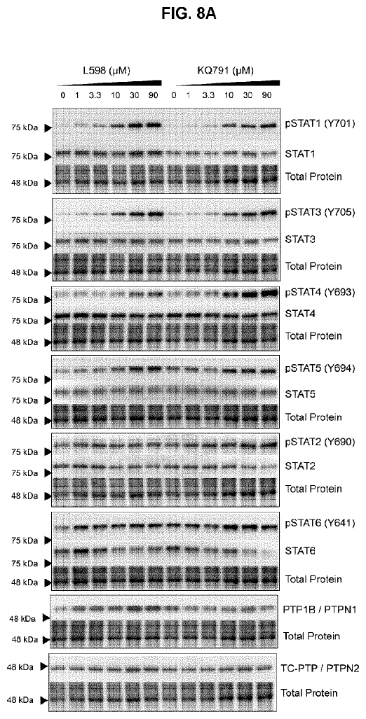

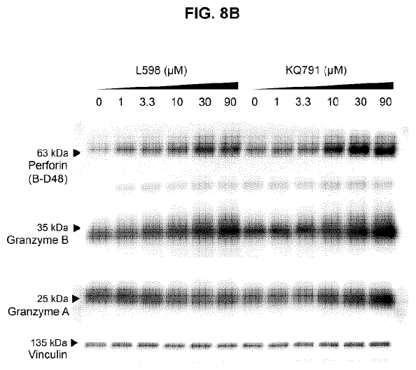

NK cell mediated direct cytolysis depends on degranulation of lytic granules at immunological synapses. To understand the molecular mechanisms behind the enhanced cytolysis functions, the lytic granule expression was investigated. It was found that at steady state, mRNA expressions of lytic granules perforin, granzyme A, granzyme B and granzyme K (data not shown) were upregulated in NK-92 cells treated with L598 at 30 μM for three days (FIG. 2A). Protein expressions of lytic granules perforin and granzyme B were also upregulated in a dosage dependent manner following gradual increasing concentration of the L598 or KQ791 treatment, whereas granzyme A protein expression was relatively stable with or without L598 treatment (FIGS. 2B and 8B).

To investigate the relationship between the increased amount of pre-formed lytic granules and enhanced tumor cytolysis upon inhibition of PTPN1 and PTPN2 enzymatic functions, the degranulation activities of NK-92 cells, with or without tumor target cell K-562 stimulations or calcium influx inducers PMA and lonomycin that bypass receptor signaling activation, was examined. As shown before, the majority of NK-92 cells from the L598 treatment group showed higher intracellular expression of perforin and granzyme B at steady state. It was observed that by the end of tumor target cell stimulations for six hours, a smaller percentage of NK-92 cells expressing both CD107a at surface and intracellular lytic granules simultaneously, which is considered as “degranulating” cells, were present in the L598 treatment group compared with the control group (FIGS. 2D and 2E). In contrast, more tumor target K-562 cells were found dead with the L598 treated NK-92 cells compared with the control cells (FIG. 2G), suggesting that a more efficient degranulation might be caused by the inactivation of PTPN1 and PTPN2. To test this hypothesis, the dynamics of degranulation rate over the three-hours cytolytic assay was investigated, and it was found that although NK-92 with or without L598 treatment followed a similar degranulation pattern (FIG. 2H), NK-92 treated with L598 degranulated perforin more rapidly, starting at 30 mins and granzyme B starting at 60 mins. In addition, the existence of a larger percentage of CD107a⁻, Perforin⁺ or CD107a⁻, Granzyme B⁺ population in the L598 treatment group, after six-hours tumor target cell stimulations, suggests that more rapid de novo synthesis of lytic granules might occur, to replenish those released lytic granules (FIGS. 2D and 2E). On the other hand, PMA and lonomycin induced almost all NK-92 cells to degranulate as most of the cells were CD107a⁺; there were more “degranulating” and less “degranulated” (CD107a⁺, Perforin⁻ or CD107a⁺, Granzyme B⁻) NK-92 from the L598 treatment group than in the control group by the end of six-hours stimulation, indicating that a more persistent degranulation occurred that might be associated with increased lytic granule contents (FIG. 2D-2F). Together, these results show that PTPN1 and PTPN2 mediated negative regulation of NK-92 anti-tumor cytolysis is related to increased expression of lytic granule perforin and granzyme B and persistent degranulation.

### Co-Inhibition of PTPN1 and PTPN2 Sensitizes NK Cells to IL-2 Activation

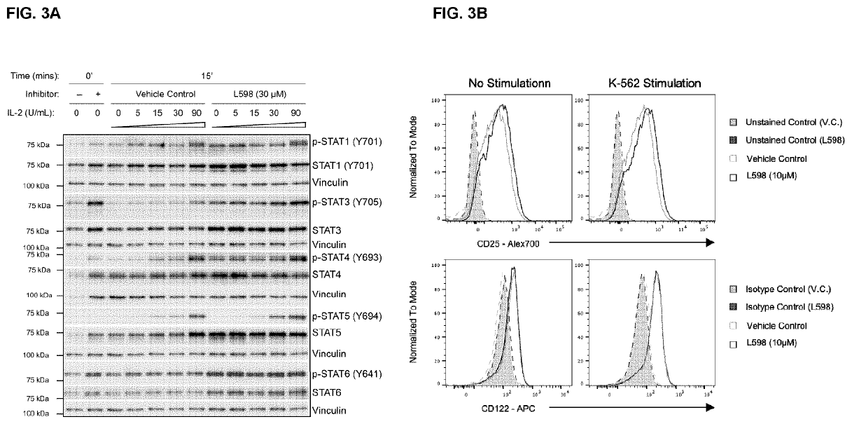

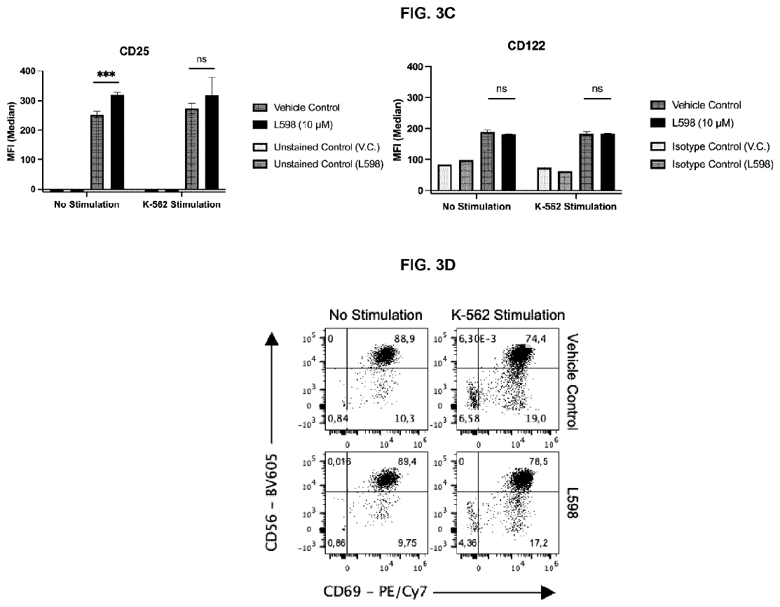

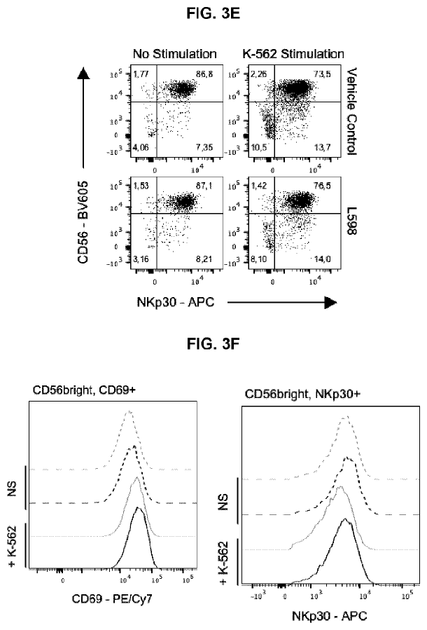

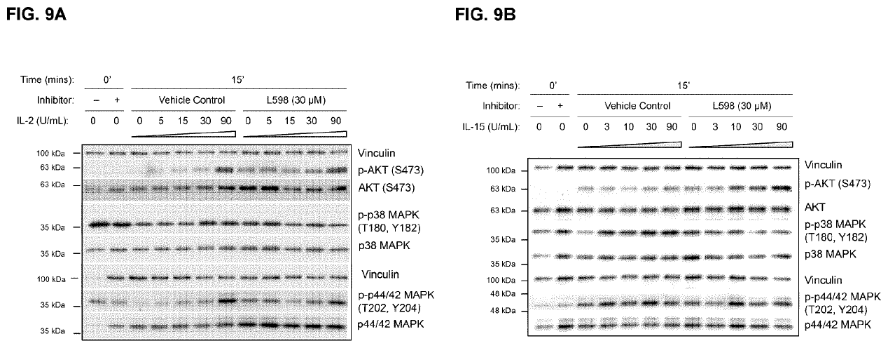

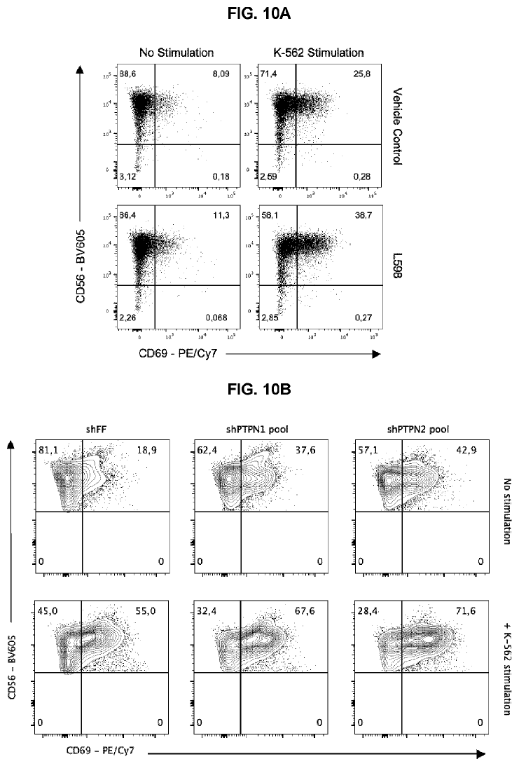

PTPN1 and PTPN2 are known direct negative regulators in cytokine induced JAK/STAT signaling pathways. In regular cell culture conditions, increased phosphorylation of STAT1, STAT3, STAT4 and STAT5 is observed in a dosage dependent manner following increased concentrations of L598 or KQ791, whereas phosphorylation of STAT2 and STAT6 were relatively stable (FIG. 8A), indicating that suppressing PTPN1 and PTPN2 enzymatic function sensitizes NK-92 cells to certain cytokine stimulations. This cytokine stimulation could be exogenously added cytokine IL-2 to maintain NK-92 cell survival and proliferation, as well as auto-secreted cytokines by NK-92 including IFN-γ. To test this hypothesis, an acute IL-2 sensitization assay was performed. After L598 treatment, NK-92 cells became more sensitive to low dosage (below 100 IU/mL) and short-term (15 mins) IL-2 stimulation as shown by dosage-dependent increasing phosphorylation of IL-2 downstream STATs members in NK cells: STAT1, STAT3, STAT4 and STAT5 but not IL-2 signaling independent STAT6 (FIG. 3A), which mimicked the activated signaling pathways identified with the previously mentioned L598 titration assay (FIG. 9A). Interestingly, unlike increased phosphorylation of STAT5 that can be detectable upon IL-2 stimulation at minimum of 30 IU/mL, basal phosphorylation of STAT1, STAT3 and STAT4 increased in NK-92 cells from the L598 treatment group and after six-hours of serum and cytokine starvations in presence of the L598, suggesting that there might be auto-secreted cytokines to maintain these activated signaling pathways at steady state (FIG. 3A). Besides, inhibition of PTPN1 and PTPN2 with L598 might upregulate total protein expressions of the corresponding STATs members (FIG. 3A), suggesting protein synthesis rate might be affected.

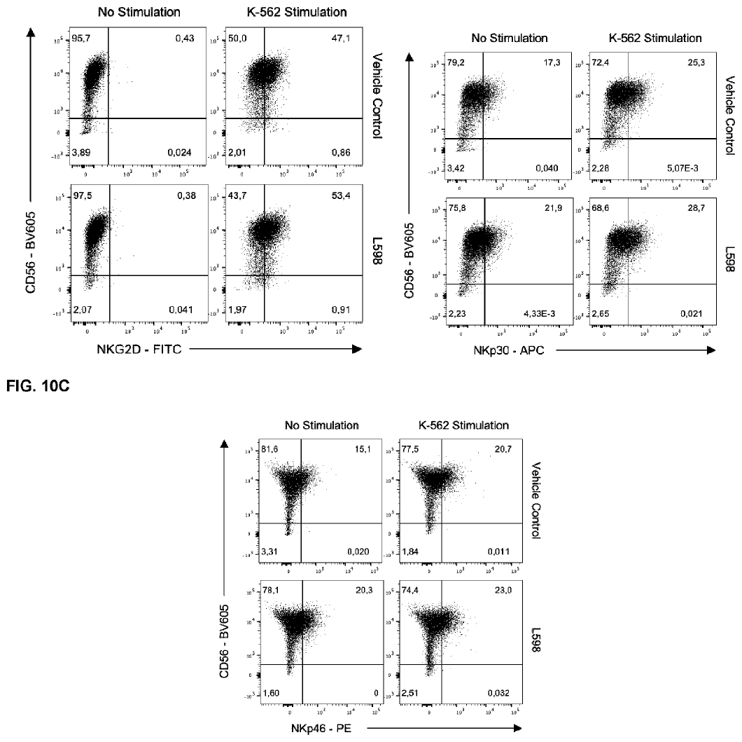

To understand why NK-92 cells became sensitized to IL-2 response, the IL-2 receptor expression was examined and it was found that IL-2 specific a subunit (CD25) of the tripartite high affinity IL-2 receptor complex was upregulated at steady state upon L598 treatment, whereas the expression of the p subunit (CD122) that are shared with IL-15 receptor complex remained stable (FIGS. 3B and 3C), indicating that sensitized IL-2 response is possible through receptor signaling. In addition, NK-92 cells (FIG. 10A) as well as cord blood isolated NK cells (FIGS. 3D-F) treated with L598 were in a more activated state with upregulation of CD69 and activating receptors NKp30, NKp46 and NKG2D (FIG. 10C) with or without tumor target cell stimulations, whose receptor signaling might further synergize with pre-set cytokine activations to further improve NK-92 cell anti-tumor cytolysis. Upregulation of CD69 was also observed in stable knockdown shPTPN1 pool (SEQ ID NOs: 21 to 24) or shPTPN2 pool (SEQ ID NOs: 25 to 28) NK-92 cells compared with shFF control (targeting Firefly luciferase from Photinus pyralis; SEQ ID NO: 29) cells (FIG. 10B). This suggests that co-inhibition of PTPN1 and PTPN2 with L598 activates NK cells through sensitized cytokine stimulation.

### Co-Inhibition of PTPN1 and PTPN2 Increases Pro-Inflammatory Cytokines Expression

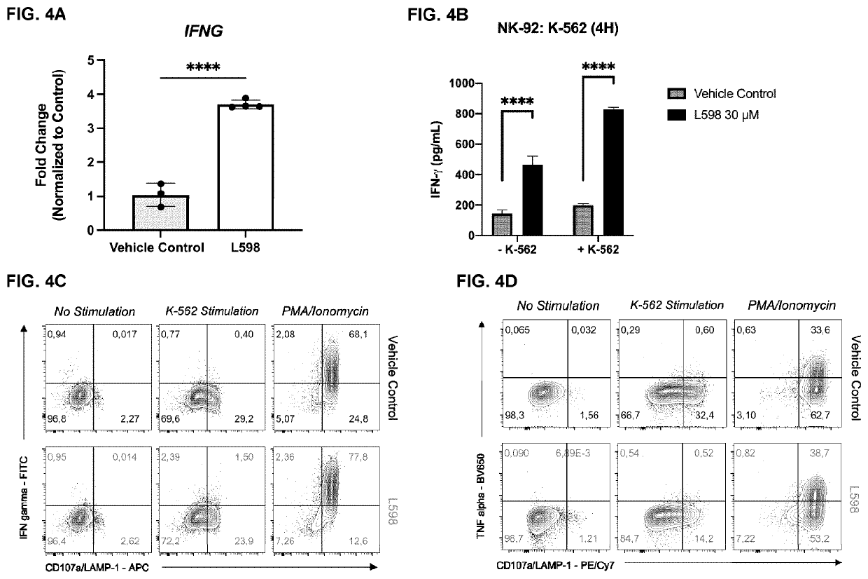

In humans, NK cells are subtyped into CD56^{bright} and CD56^{dim} populations with specialized functions in cytokine secretions and direct cytolysis, respectively. NK-92 is a transformed cell line that expresses CD56^{bright} and mimics activated NK cells with cytolysis functions against a broad spectrum of tumor cells. Therefore, the cytokine secretion functions of NK-92 cells upon tumor target cells stimulations with a focus on Th1 response pro-inflammatory cytokines IFN-γ and TNF-α was studied. At steady state, an upregulation of IFN-γ mRNA in the L598 treatment group was observed while these cells also secreted significantly more IFN-γ proteins than the control group with or without tumor target cell stimulations (FIGS. 4A and 4B). Upon tumor cell or PMA and ionomycin stimulations, NK-92 cells degranulated as shown by upregulated expression of CD107a, and they also produced IFN-γ and TNF-α (FIGS. 4C and 4D). As larger percentages of CD107a⁺, IFN-γ⁺ and CD107a⁺, TNF-α⁺ cells were found in the L598 treatment group, it was suspected that inhibition of PTPN1 and PTPN2 with L598 has multi-functional results in improving NK-92 anti-tumor cytolysis.

### Co-Inhibition of PTPN1 and PTPN2 Enhanced Cytolysis is Reversible in Relation to Cytolytic Granule Production

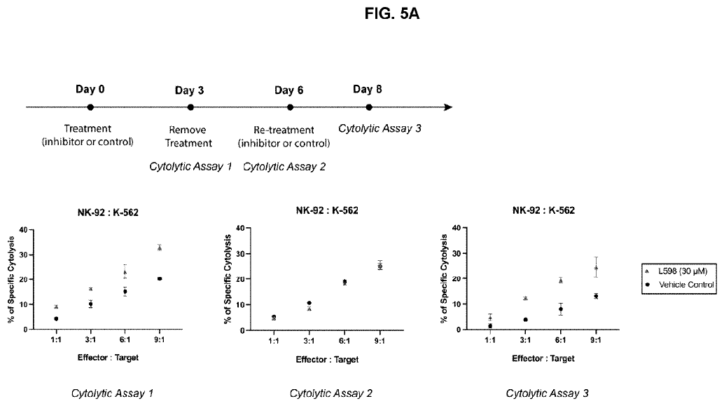

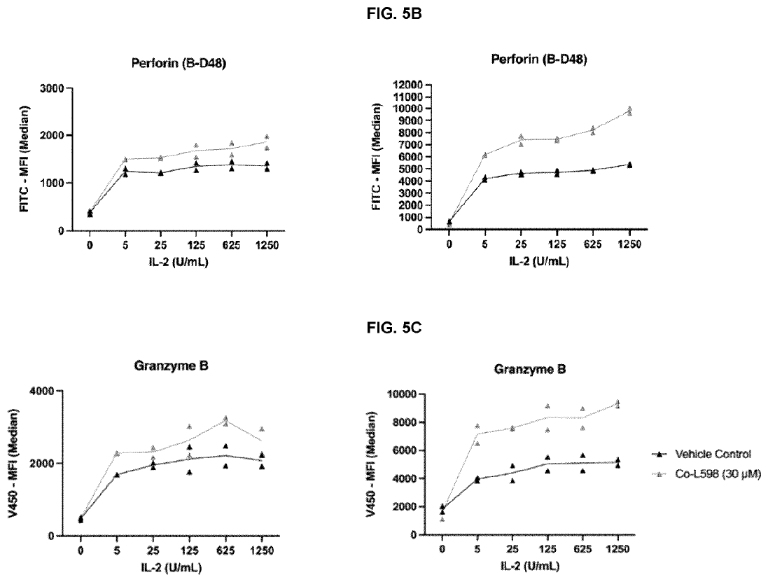

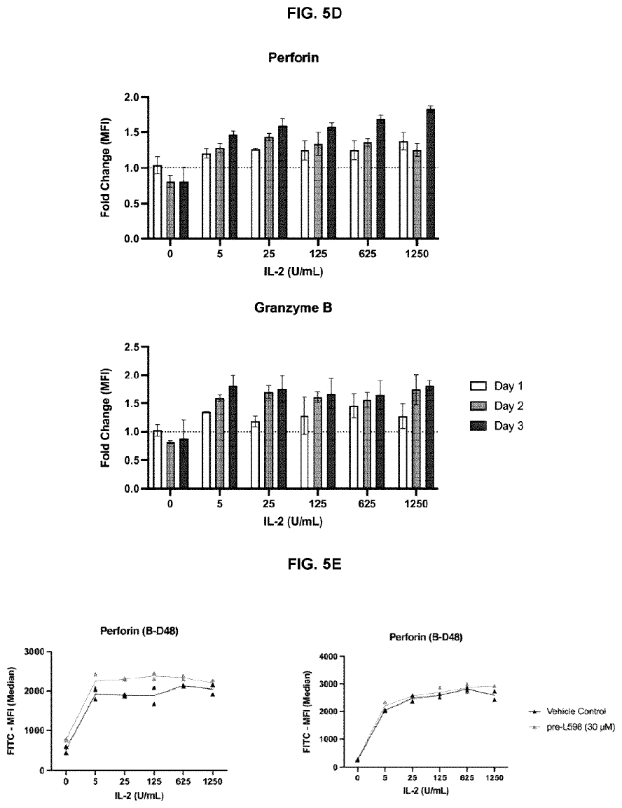

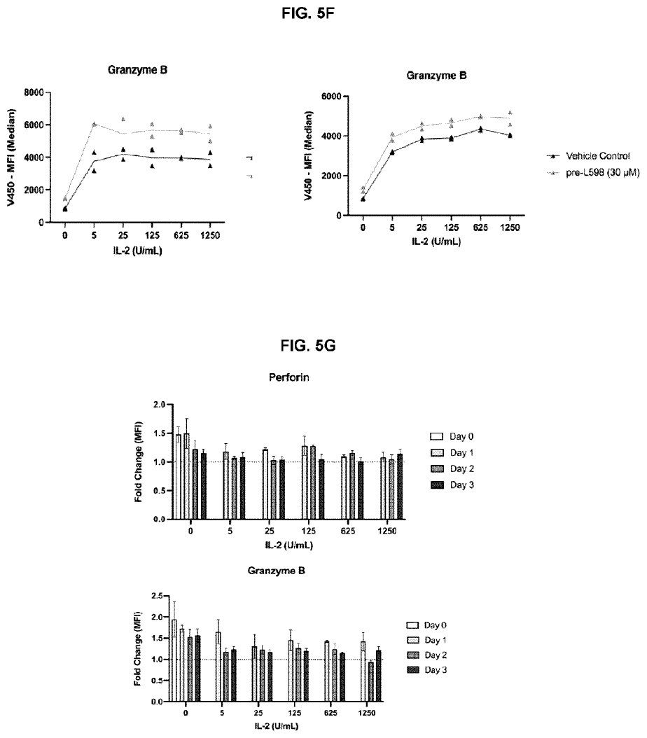

The inhibition of PTPN1 and PTPN2 with L598 enhances NK cell cytolysis against susceptible target cells K-562. This enhanced cytolysis function is reversible when the PTPN1 and PTPN2 inhibitor L598 is withdrawn for three days after the initial treatment. Also, an additional two-days treatment with L598 at the same molar concentration once more improved the anti-tumor cytolysis function to a similar level of the initial treatment (FIG. 5A). It was hypothesized that the reversible effector function is related to the reversible expression of pre-formed cytolytic granules as shown in FIG. 2. To examine this hypothesis and the relationship between sensitized IL-2 response and cytolytic granule expression, an IL-2 titration assay in presence of L598 for three consecutive days was performed. The results showed that a one-day treatment with L598 started improving the fold change of intracellular expression of perforin and granzyme over the control group. By day three, an approximately two-fold increase of these lytic granules was observed with IL-2 stimulations at 1250 IU/mL (FIG. 5D). In addition to the correlation between lytic granule production and IL-2 concentration, it was observed that with L598 treatment, the thresholds of maximal expression of perforin and granzyme B were also upregulated (FIGS. 5B and 5C).

Interestingly, as shown in FIG. 5A, withdrawing of L598 for three consecutive days reduced the fold change of perforin and granzyme B to around one-fold and the maximal expression of these lytic granules were similar (FIGS. 5E-G), Of note, there was almost no difference in granzyme B expression between the treatment and control group and low expression of perforin without IL-2 stimulations, suggesting that phosphorylation of PI3K/AKT mediated IL-2 survival signal might act as a pre-request in increased lytic granule productions (FIGS. 9A and B). Taken together, these results unexpectedly indicate that NK-92 cells were sensitive to STAT mediated IL-2 activation that was associated with increased cytolytic granule production at steady state. In addition, these results also unexpectedly suggest that both of PTPN1 and PTPN2 are potential targets in NK cell related anti-tumor immunotherapy while PTPN1 and PTPN2 enzymatic regulation of cytolysis function is under quantitative, specific and dynamic regulation.

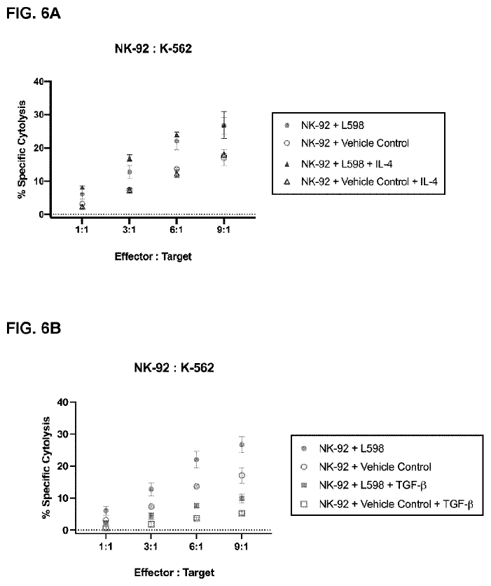

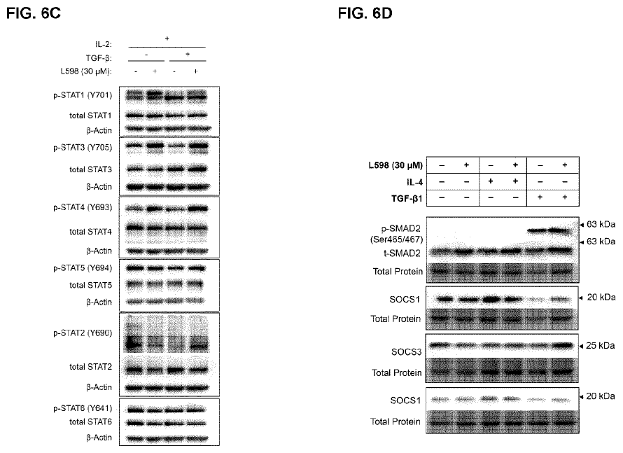

To test whether co-inhibition of PTPN1 and PTPN2 sensitized NK cell anti-tumor cytolysis function is inhibited by immune regulatory cytokines TGF-β1 or Th2 cytokine IL-4, NK-92 cells were pre-treated with L598 or vehicle control in regular cell media supplemented with IL-2 in combination with TGF-β1 or IL-4 for three days. Notably, addition of IL-4 did not reduce nor improve NK-92 cells cytolysis function against K-562 cells with or without L598 treatment (FIG. 6A). On the other hand, addition of TGF-β1 significantly reduced NK-92 cytolysis functions; however, with L598 treatment, these NK-92 cells continued exhibiting enhanced anti-tumor cytolysis against K-562 compared with the vehicle control group (FIG. 6B). Interestingly, under TGF-β1 and IL-2 stimulation condition, L598 treated NK-92 cells retained enhanced phosphorylation of STAT1, STAT3 and STAT4 signaling (FIG. 6C), which was like the IL-2 sensitization response as shown previously (FIG. 3A). Furthermore, TGF-β1 activated phosphorylation of SMAD2 did not change significantly with L598 treatment while expressions of SOCS1 and SOCS3 that act in a negative feedback to attenuate IL-2 and/or IL-4 signaling, did not change with L598 treatment (FIG. 6D). Taken together, these results surprisingly indicated that co-inhibition of PTPN1 and PTPN2 enhanced NK cell cytolysis function is resistant at certain level to the immune suppressive tumor microenvironment possibly through sensitized JAK/STAT signaling pathway that does not affect signal termination and is independent of phosphor-SMAD signaling pathway.

### Example 3

### Discussion

PTPN1 and PTPN2 are prototypes of non-receptor protein tyrosine phosphatases and have been studied in adaptive and innate immune cells: T cells, B cells, macrophages, dendritic cells development and functions. PTPN1 has also been investigated in neutrophils, eosinophils and mast cells where PTPN2's functional role has not been revealed yet. The role of these two phosphatases, together or separately, in NK cell development and cytolytic functions is unknown. The results above show that co-inhibition of PTPN1 and PTPN2 with the inhibitor compounds L598 or KQ791 enhanced NK cell cytolysis activities against sensitive tumor target cells but had no effect to resistant target cells, indicating that these phosphatases are novel negative regulators in potentiating NK effector functions, but that they are under control for non-specifically attacking bystander cells. Genetically knocking-down PTPN1 and PTPN2 expression individually in NK-92 cells (FIG. 1D) showed that inhibition of PTPN1 was more potent in improving cytolytic functions than PTPN2. This may be caused by the relatively high expression of PTPN1 mRNA and protein levels relative to PTPN2 in NK-92 cells, as their abundances might affect the amount and types of substrates binding, and they are highly homologous proteins that might share redundant functions (FIG. 1B). However, this does not rule out the possibility that PTPN1 regulates central type(s) of cytokine induced specific JAK/STAT signaling pathways that are not regulated by PTPN2.

Heterogeneity in NK cell lytic granules have been described in early studies shown by electron microscopy examinations: type I granule (50-700 nm) is filled with a dense core surrounded by a thin layer of vesicle, type II granule (200-1000 nm) is filled with multiple vesicles and membrane whorls, and intermediate granules that transit between type I and type 1l. However, it is not known how the content of the lytic granule differs. The results above showed that granzyme B and perforin protein expressions are more prominently affected by inhibition of PTPN1 and PTPN2 with L598 or KQ791, whereas granzyme A expression is relatively stable despite its mRNA level being upregulated (FIGS. 2 A-B). The possibility of existence of different types of granules may indicate that the enhanced tumor target cells death observed is mainly due to granzyme B activities.

Depending on signal strength, IL-2 has different functional impacts on immune cells. One example is CD8⁺ T cell differentiation. High dosage of IL-2 supports effector T cell differentiation through high expression of Blimp-1 and T-bet resulting in increased expression of granzyme B and perforin whereas low dosage of IL-2 supports memory T cell differentiation through decreased expression of Blimp-1 and increased expressions of Bcl-6, IL-7Rα and Eomes. In NK cells, differential responses to IL-2 dosage have not been distinguished yet. The results presented above show a strengthened NK cell effector response to low dosage of IL-2 (FIG. 3A). Recent epigenetic studies have demonstrated that de novo formation of STAT1, STAT3, STAT4 and STAT5 binding sites upon acute cytokine IL-2 and IL-12 stimulations characterizes formation of innate memory in activated NK cells. By screening JAK/STAT signaling pathways with dosage dependent inhibition of PTPN1 and PTPN2 with L598 or KQ791 for three days in regular cell culture condition (FIG. 8A), gradually increased tyrosine phosphorylation of STAT1, STAT3, STAT4 and STAT5 was observed, suggesting that these NK cells acquire innate memory features. CAR-modified memory-like NK cells have shown potent response to lymphoma and the present results unexpectedly support another way to induce memory-like NK cells that could be applied to CAR-NK anti-tumor immunotherapy.

The above results show that the enhanced cytolytic functions is reversible upon withdrawal of the PTPN1 and PTPN2 inhibitor L598 which suggests dynamic changes in cytolytic granule perforin and granzyme B expression (FIG. 5). The reversible effects in tumor target cytolysis and lytic granule expression may be linked to the reversible phosphatases activities of PTPN1 and PTPN2, occurring early in the signaling pathways upon cell activation while the downstream initiated transcriptional and/or translational changes are temporary and less likely to be the results from stable changes in chromatin structures that are involved in irreversible/terminal cell differentiation. Furthermore, the augmented expression of perforin and granzyme B upon IL-2 stimulation is closely dependent on co-inhibition of PTPN1 and PTPN2 while IL-2 is the primary stimulator of perforin and granzyme B expression. Indeed, cytokine IL-15, IL-21 alone or in combination of cytokine IL-12, IL-18, IL-27 have also been reported to induce cytolytic granule expression in NK cells, indicating that PTPN1 and PTPN2 have potential roles in regulating their signaling pathway as well. The reversible effects also indicate that augmented cytolytic functions is less likely to induce hyper-inflammation and NK cell tumor transformation that can result from constitutively activated JAK/STAT under chronic inhibition of PTPN1 and PTPN2 background.

In summary, PTPN1 and PTPN2 have been unexpectedly shown to be novel targets to potentiate NK cell anti-tumor cytolytic functions by increasing cytolytic granule productions and proinflammatory cytokines production after cell activation. PTPN1 and PTPN2 are possible tonic negative regulators of IL-2 signaling acting at the signal initiation stage whereas other identified negative regulators such as SOCS3 acts as an IL-2 signal terminator that mediated polyubiquitin tagged JAK/STAT proteins degradation. Inhibition of PTPN1 and PTPN2 sensitizes NK cells to cytokine stimulations at low dosage, which may lead to innate memory features formation. Pharmacological inhibition of PTPN1 and PTPN2 enhanced NK cell effector function is reversible, indicating their safe application to improve current CAR-NK anti-tumor immunotherapy.

While preferred embodiments have been described above and illustrated in the accompanying drawings, it will be evident to those skilled in the art that modifications may be made without departing from this disclosure. Such modifications are considered as possible variants comprised in the scope of the disclosure.

SHRNA SEQUENCES

| SEQ ID NO: | Sequence | Description |
|---|---|---|
| SEQ ID NO: 21 | TGCTGTTGACAGTGAGCGCTCTGCTAGATCTAGTTCTC | shPTPN1 (1) |
| | AATAGTGAAGCCACAGATGTATTGAGAACTAGATCTAG | |
| | CAGATTGCCTACTGCCTCGGA | |
| SEQ ID NO: 22 | TGCTGTTGACAGTGAGCGACCGGTTCACCTTGCCGAG | shPTPN1 (2) |
| | AGATAGTGAAGCCACAGATGTATCTCTCGGCAAGGTGA | |
| | ACCGGGTGCCTACTGCCTCGGA | |
| SEQ ID NO: 23 | TGCTGTTGACAGTGAGCGAACGGACGTTGGTTCTGCA | shPTPN1 (3) |
| | CTATAGTGAAGCCACAGATGTATAGTGCAGAACCAACG | |
| | TCCGTCTGCCTACTGCCTOGGA | |
| SEQ ID NO: 24 | TGCTGTTGACAGTGAGCGCCCCTGTTATCTGCTAGATC | shPTPN1 (4) |
| | TATAGTGAAGCCACAGATGTATAGATCTAGCAGATAAC | |
| | AGGGATGCCTACTGCCTCGGA | |
| SEQ ID NO: 25 | TGCTGTTGACAGTGAGCGCTGACCAAGAGATGCTGTTT | shPTPN2 (1) |
| | AATAGTGAAGCCACAGATGTATTAAACAGCATCTCTTG | |
| | GTCATTGCCTACTGCCTOGGA | |
| SEQ ID NO: 26 | TGCTGTTGACAGTGAGCGCACAGTACATCTACTACAAT | shPTPN2 (2) |
| | TATAGTGAAGCCACAGATGTATAATTGTAGTAGATGTAC | |
| | TGTATGCCTACTGCCTCGGA | |
| SEQ ID NO: 27 | TGCTGTTGACAGTGAGCGCACGATGGAAAGAACTTTCT | shPTPN2 (3) |
| | AATAGTGAAGCCACAGATGTATTAGAAAGTTCTTTCCAT | |
| | CGTTTGCCTACTGCCTOGGA | |
| SEQ ID NO: 28 | TGCTGTTGACAGTGAGCGACCGCTGTACTTGGAAATTC | shPTPN2 (4) |
| | GATAGTGAAGCCACAGATGTATCGAATTTCCAAGTACA | |
| | GCGGCTGCCTACTGCCTOGGA | |
| SEQ ID NO: 29 | TGCTGTTGACAGTGAGCGACCGCCTGAAGTCTCTGATT | shFireFly (or |
| | AATAGTGAAGCCACAGATGTATTAATCAGAGACTTCAG | shFF) |
| | GCGGGTGCCTACTGCCTCGGA | |

1. An ex vivo method of stimulating an isolated natural killer (NK) cell comprising:
treating said isolated natural killer (NK) cell with an effective amount of a compound of structural Formula I, of structural Formula II, or pharmaceutically acceptable salts thereof, and stereoisomers thereof, or combinations thereof: 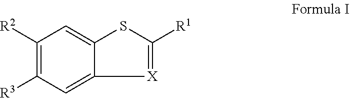
wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —ON, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN: (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₃ hydroxyalkyl, (viii) aryl or heteroaryl wherein the aryl or heteroaryl group itself may be optionally substituted by 1-3 halogens, C₁₋₃ alkyl or C₁₋₃ haloalkyl; and
x is an integer from 0 to 2; 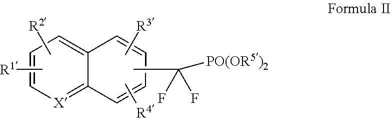
wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
each R^{5′} group is independently selected from the group consisting of H and C₁₋₃alkyl optionally substituted with 1-3 halogens, and
x is 0, 1, or 2,
to obtain a stimulated isolated NK cell.

2. The ex vivo method of claim 1, wherein said compound of Formula I is of structural Formula Ia, or a pharmaceutically acceptable salts thereof, and stereoisomers thereof: 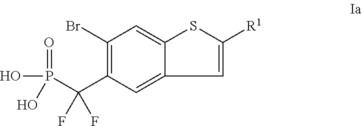
wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —ON: (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

3. The ex vivo method of claim 1, wherein said compound of formula I is of structural Formula Ib, or a pharmaceutically acceptable salts thereof, and stereoisomers thereof: 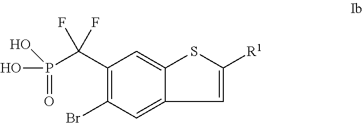
wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN: (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁a alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

4. The ex vivo method of claim 1, wherein said compound is a compound selected from the following compounds:

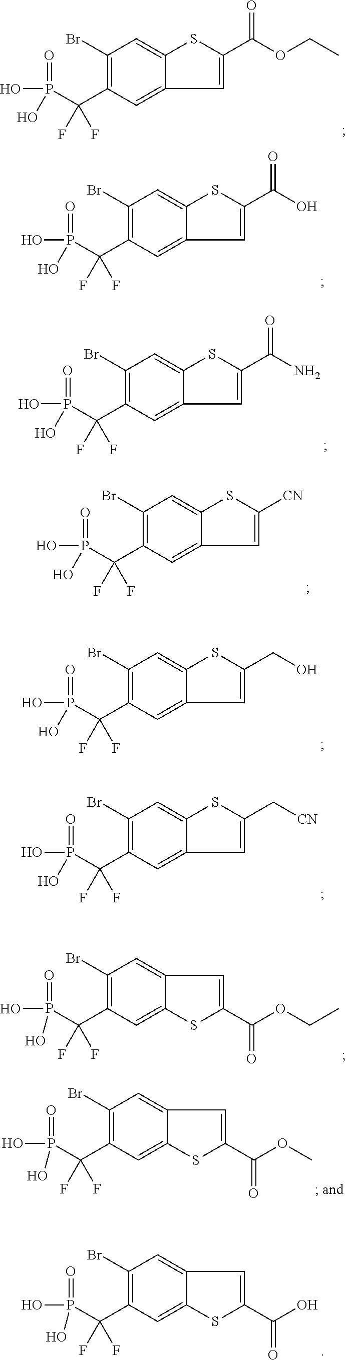

5. The ex vivo method of claim 1, wherein said compound of formula II is of structural Formula IIa, or a pharmaceutically acceptable salts thereof, and stereoisomers thereof: 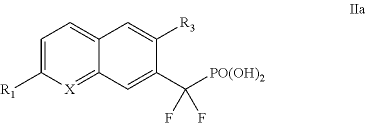
wherein
X is selected from CH and N;
R¹ is selected from the group consisting of (a) C₁₋₃ alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR⁶, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂ alkyl optionally substituted with 1-3 F, (l) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂ alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R³ is halogen;
R⁶ is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, and (v) —OC₁₋₃alkyl optionally substituted with 1-3 halogens; and
x is 0, 1, or 2.

6. The ex vivo method of claim 5, wherein the compound is selected from the following compounds:

| Ex. | Structure |
|---|---|
| 2 | 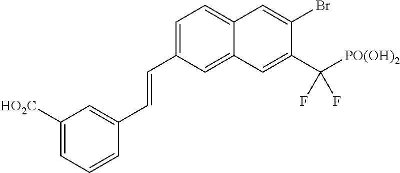 |
| 3 | 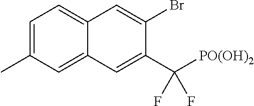 |
| 4 | 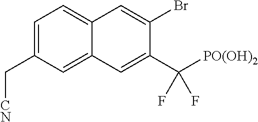 |
| 5 | 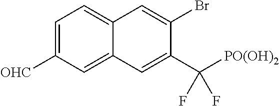 |
| 6 | 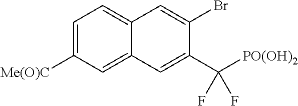 |
| 8a | 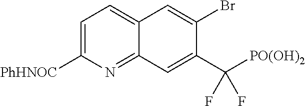 |
| 8b | 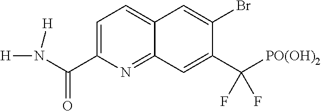 |
| 8c | 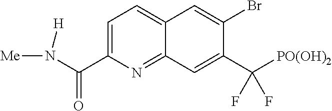 |
| 8d | 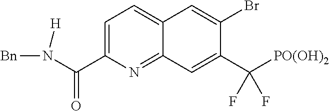 |
| 8e | 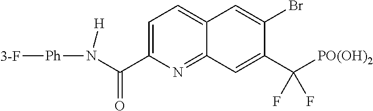 |
| 9a | 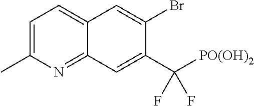 |
| 9c | 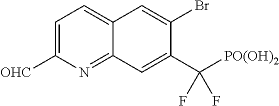 |
| 9d | 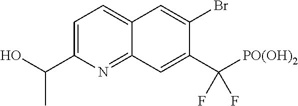 |
| 9e | 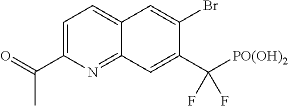 |
| 9f | 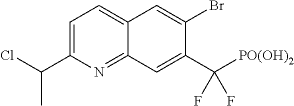 |
| 9g | 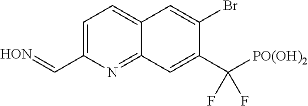 |
| 9h | 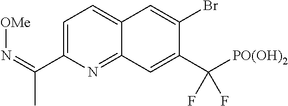 |
| L598 | Br |

or a pharmaceutically acceptable salt thereof.

7. The ex vivo method of claim 2, wherein the compound of formula (Ia) is 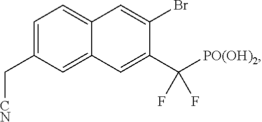
or a pharmaceutically acceptable salt thereof.

8. The method of claim 1, further comprising a stimulation with interleukin-2 (IL-2).

9. A stimulated isolated NK cell prepared by the ex vivo method of claim 1.

10. A composition comprising the stimulated isolated NK cell of claim 9 and a pharmaceutically acceptable carrier.

11. The ex vivo method of claim 1, wherein said isolated natural killer (NK) cell is isolated from a human subject.

12. (canceled)

13. The ex vivo method of claim 1, wherein said isolated natural killer (NK) cell further comprises a chimeric antigen receptor (CAR).

14. A method of treating cancer comprising administering to a subject in need thereof a therapeutically effective amount of the stimulated isolated NK cell of claim 9.

15.-16. (canceled)

17. The method of claim 14, wherein said cancer is selected from the group consisting of prostate cancer, breast cancer, brain cancer, glioma, lung cancer, salivary cancer, stomach cancer, thymic epithelial cancer, thyroid cancer, ovarian cancer, multiple myeloma, leukemia, melanoma, lymphoma, gastric cancer, kidney cancer, pancreatic cancer, bladder cancer, colon cancer and liver cancer.

18. The method of claim 14, further comprising the administration of one or more additional compounds selected from the group consisting of:
(a) a cytotoxic agent;
(b) an antimetabolite;
(c) an alkylating agent;
(d) an anthracycline;
(e) an antibiotic;
(f) an anti-mitotic agent;
(g) an hormone therapy;
(h) a signal transduction inhibitor;
(i) a gene expression modulator;
(k) an apoptosis inducer;
(k) an angiogenesis inhibitor
(l) an immunotherapy agent.

19. The method of claim 18, wherein:
said cytotoxic agent is selected from the group consisting of taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, analogs or homologs thereof, and combinations thereof,
said antimetabolites is selected from the group consisting of methotrexate, 6-mercaptopurine, 6-thioguanine, gemcitabine, cytarabine, 5-fluorouracil decarbazine, and combinations thereof;
said alkylating agent is selected from the group consisting of mechlorethamine, thioepa chlorambucil, melphalan, carmustine (BSNU), lomustine (CCNU), cyclophosphamide, busulfan, dibromomannitol, streptozotocin, mitomycin C, cis-dichlorodiamine platinum (II) (DDP) cisplatin, and combinations thereof;
said anthracycline is selected from the group consisting of daunorubicin, doxorubicin, and combinations thereof;
said antibiotic is selected from the group consisting of dactinomycin, bleomycin, mithramycin, anthramycin (AMC), and combinations thereof;
said anti-mitotic agent is selected from the group consisting of vincristine, vinblastine, and combinations thereof;
said signal transduction inhibitor is selected from the group consisting of imatinib, trastuzumab, PARPI, CDKI and combinations thereof;
said gene expression modulator is selected from the group consisting of a siRNA, a shRNA, and antisense oligonucleotide, an HDAC inhibitor, and combinations thereof;
said immunotherapy agent is selected from the group consisting of a monoclonal antibody, a dendritic cell (DC) vaccine, an antigen therapy, and combinations thereof;
said hormone therapy is a luteinizing hormone-releasing hormone (LHRH) antagonist;
said apoptosis inducers is a recombinant human TNF-related apoptosis-inducing ligand (TRAIL);
said angiogenesis inhibitors is selected from the group consisting of sorafenib, sunitinib, pazopanib, everolimus and combinations thereof; and
said monoclonal antibody is selected from the group consisting of anti-CTLA4, anti-PD1, anti-PD-L1, anti-LAG3, anti-KIR, and combinations thereof.

20.-31. (canceled)

32. The method claim 14, wherein said stimulated isolated NK cell is an autologous isolated NK cell from said patient in need thereof.


SEQUENCE LISTING (29 sequences)

SEQ ID NO: 1 - DNA, 20 bp, Homo sapiens
  source 1..20: mol_type = other DNA
agtatgcctgccgtgtgaac

SEQ ID NO: 2 - DNA, 20 bp, Homo sapiens
  source 1..20: mol_type = other DNA
tcatccaatccaaatgcggc

SEQ ID NO: 3 - DNA, 20 bp, Homo sapiens
  source 1..20: mol_type = other DNA
aggacctctcgaagtgttgg

SEQ ID NO: 4 - DNA, 21 bp, Homo sapiens
  source 1..21: mol_type = other DNA
ggctttgtatttggcttttcc

SEQ ID NO: 5 - DNA, 20 bp, Homo sapiens
  source 1..20: mol_type = other DNA
ttaacttcgcttccgctggc

SEQ ID NO: 6 - DNA, 20 bp, Homo sapiens
  source 1..20: mol_type = other DNA
tgcgctggaactcgtctcac

SEQ ID NO: 7 - DNA, 20 bp, Homo sapiens
  source 1..20: mol_type = other DNA
ggcccgtcatggagatggaa

SEQ ID NO: 8 - DNA, 21 bp, Homo sapiens
  source 1..21: mol_type = other DNA
gcttggccactctacatggga

SEQ ID NO: 9 - DNA, 20 bp, Homo sapiens
  source 1..20: mol_type = other DNA
tcgagcgggagttcgaagag

SEQ ID NO: 10 - DNA, 22 bp, Homo sapiens
  source 1..22: mol_type = other DNA
cacgactgtgatcatatgggct

SEQ ID NO: 11 - DNA, 21 bp, Homo sapiens
  source 1..21: mol_type = other DNA
cgcctacctcaggcttatctc

SEQ ID NO: 12 - DNA, 19 bp, Homo sapiens
  source 1..19: mol_type = other DNA
cctcgacagtcaggcagtc

SEQ ID NO: 13 - DNA, 23 bp, Homo sapiens
  source 1..23: mol_type = other DNA
tcaggttgattgatgtgggacag

SEQ ID NO: 14 - DNA, 25 bp, Homo sapiens
  source 1..25: mol_type = other DNA
gaccatgtagggtcttgaatgagga

SEQ ID NO: 15 - DNA, 21 bp, Homo sapiens
  source 1..21: mol_type = other DNA
tgggggacccagagattaaaa

SEQ ID NO: 16 - DNA, 22 bp, Homo sapiens
  source 1..22: mol_type = other DNA
tttcgtccataggagacaatgc

SEQ ID NO: 17 - DNA, 24 bp, Homo sapiens
  source 1..24: mol_type = other DNA
atcaacacatttcatctgggcttc

SEQ ID NO: 18 - DNA, 22 bp, Homo sapiens
  source 1..22: mol_type = other DNA
aaacgtgatgtccgccatactg

SEQ ID NO: 19 - DNA, 20 bp, Homo sapiens
  source 1..20: mol_type = other DNA
aagtgatggctgaactgtcg

SEQ ID NO: 20 - DNA, 20 bp, Homo sapiens
  source 1..20: mol_type = other DNA
gcaggcaggacaaccattac

SEQ ID NO: 21 - DNA, 97 bp, Homo sapiens
  source 1..97: mol_type = other DNA
tgctgttgacagtgagcgctctgctagatctagttctcaatagtgaagccacagatgtattgagaactagatctagcagattgcctactgcctcgga

SEQ ID NO: 22 - DNA, 97 bp, Homo sapiens
  source 1..97: mol_type = other DNA
tgctgttgacagtgagcgaccggttcaccttgccgagagatagtgaagccacagatgtatctctcggcaaggtgaaccgggtgcctactgcctcgga

SEQ ID NO: 23 - DNA, 97 bp, Homo sapiens
  source 1..97: mol_type = other DNA
tgctgttgacagtgagcgaacggacgttggttctgcactatagtgaagccacagatgtatagtgcagaaccaacgtccgtctgcctactgcctcgga

SEQ ID NO: 24 - DNA, 97 bp, Homo sapiens
  source 1..97: mol_type = other DNA
tgctgttgacagtgagcgcccctgttatctgctagatctatagtgaagccacagatgtatagatctagcagataacagggatgcctactgcctcgga

SEQ ID NO: 25 - DNA, 97 bp, Homo sapiens
  source 1..97: mol_type = other DNA
tgctgttgacagtgagcgctgaccaagagatgctgtttaatagtgaagccacagatgtattaaacagcatctcttggtcattgcctactgcctcgga

SEQ ID NO: 26 - DNA, 97 bp, Homo sapiens
  source 1..97: mol_type = other DNA
tgctgttgacagtgagcgcacagtacatctactacaattatagtgaagccacagatgtataattgtagtagatgtactgtatgcctactgcctcgga

SEQ ID NO: 27 - DNA, 97 bp, Homo sapiens
  source 1..97: mol_type = other DNA
tgctgttgacagtgagcgcacgatggaaagaactttctaatagtgaagccacagatgtattagaaagttctttccatcgtttgcctactgcctcgga

SEQ ID NO: 28 - DNA, 97 bp, Homo sapiens
  source 1..97: mol_type = other DNA
tgctgttgacagtgagcgaccgctgtacttggaaattcgatagtgaagccacagatgtatcgaatttccaagtacagcggctgcctactgcctcgga

SEQ ID NO: 29 - DNA, 97 bp, Photinus pyralis
  source 1..97: mol_type = other DNA
tgctgttgacagtgagcgaccgcctgaagtctctgattaatagtgaagccacagatgtattaatcagagacttcaggcgggtgcctactgcctcgga